(19) United States
(12) Patent Application Publication
(10) Pub. No.: US 2007/0293735 A1
(43) Pub. Date: Dec. 20, 2007

(54) COGNITIVE TRAINING USING ONE OR MORE STIMULUS STREAMS AND TARGET ATTRIBUTE MATCHING

(75) Inventors: Samuel C. Chan, Alameda, CA (US); Joseph L. Hardy, Richmond, CA (US); Michael M. Merzenich, San Francisco, CA (US)

Correspondence Address: HUFFMAN LAW GROUP, P.C., 1900 MESA AVE., COLORADO SPRINGS, CO, 80906, US

(73) Assignee: Posit Science Corporation, San Francisco, CA (US)

(21) Appl. No.: 11/760,435

(22) Filed: Jun. 8, 2007

Related U.S. Application Data
(60) Provisional application No. 60/804,427, filed on Jun. 9, 2006.
(60) Provisional application No. 60/868,839, filed on Dec. 6, 2006.

(51) Int. Cl. A61B 5/00

(52) U.S. Cl. 600/300

(57) ABSTRACT

System and method for enhancing cognition using continuous performance with respect to one or more stimulus streams. At least one target attribute is presented to the subject after which a continuous sequence of stimulus groups from a stimulus set is presented one stimulus group at a time, each group including one or more stimuli, presented for a specified duration, and separated by a specified inter-stimulus-interval (ISI). For each stimulus group in the sequence: the subject is required to respond to the group, indicating when all the stimuli in the group correspond to the target attribute(s); correctness/incorrectness of the response is determined, and the duration and/or the ISI adjusted based on the determining using an adaptive procedure, e.g., a continuous performance maximum likelihood procedure. The presenting the target attribute(s), presenting the continuous sequence, and requiring/determining/adjusting for each group, are repeated iteratively to improve the cognition of the subject.

### CROSS REFERENCE TO RELATED APPLICATION(S)

This application claims the benefit of the following US Provisional Patent Applications, which are incorporated herein in their entirety for all purposes:

| Docket No. | Serial No. | Filing Date: | Title: |
|---|---|---|---|
| PS.0128 | 60/804,427 | 6/9/06 | Aristotle 1-3 |
| PS.0131 | 60/868,839 | 12/6/06 | COMPUTER BASED TRAINING |
| | | | PROGRAM TO IMPROVE |
| | | | SUSTAINED ATTENTION, |
| | | | INHIBITION, WORKING MEMORY |

The following applications are related to the present application, and are hereby incorporated by reference in their entirety for all purposes:

| | | | |
|---|---|---|---|
| PS.0216 | ******* | ******* | COGNITIVE TRAINING USING A |
| | | | CONTINUOUS PERFORMANCE |
| | | | ADAPTIVE PROCEDURE |
| PS.0232 | ******* | ******* | COGNITIVE TRAINING USING |
| | | | MULTIPLE STIMULUS STREAMS |
| | | | WITH RESPONSE INHIBITION |

### FIELD OF THE INVENTION

This invention relates in general to the use of brain health programs utilizing brain plasticity to enhance human performance and correct neurological disorders, and more specifically, to a method for improving cognition using stimulus streams.

### BACKGROUND OF THE INVENTION

Almost every individual has a measurable deterioration of cognitive abilities as he or she ages. The experience of this decline may begin with occasional lapses in memory in one's thirties, such as increasing difficulty in remembering names and faces, and often progresses to more frequent lapses as one ages in which there is passing difficulty recalling the names of objects, or remembering a sequence of instructions to follow directions from one place to another. Typically, such decline accelerates in one's fifties and over subsequent decades, such that these lapses become noticeably more frequent. This is commonly dismissed as simply “a senior moment” or “getting older.” In reality, this decline is to be expected and is predictable. It is often clinically referred to as “age-related cognitive decline,” or “age-associated memory impairment.” While often viewed (especially against more serious illnesses) as benign, such predictable age-related cognitive decline can severely alter quality of life by making daily tasks (e.g., driving a car, remembering the names of old friends) difficult.

In many older adults, age-related cognitive decline leads to a more severe condition now known as Mild Cognitive Impairment (MCI), in which sufferers show specific sharp declines in cognitive function relative to their historical lifetime abilities while not meeting the formal clinical criteria for dementia. MCI is now recognized to be a likely prodromal condition to Alzheimer's Disease (AD) which represents the final collapse of cognitive abilities in an older adult. The development of novel therapies to prevent the onset of this devastating neurological disorder is a key goal for modern medical science.

As a specific example, the ability to accurately analyze and integrate multiple information input is degraded with advanced age, especially when one or more rules are assigned to the processing of the information. This deficit occurs due to a reduction in the overall capacity of the analytical and organizational processing systems. Additionally, the complexity and number of rules add to the already demanding task of encoding multiple targets for parallel analysis. The consequences of this can be observed in reduced proficiency in organizational processing, accuracy in analytical computation and increased decision-making time.

Many situations require one to examine various aspects of a given condition to determine the right or safe course of action. A loss or decline in this ability can have adverse, even disastrous, effects, as in the case of medication intake. Additionally complex problem solving requires decomposing the problem, analyzing the pieces, synthesizing and organizing them into new solutions. Improving the ability to perform each component of the process may result in an overall efficacy in problem solving capabilities.

The majority of the experimental efforts directed toward developing new strategies for ameliorating the cognitive and memory impacts of aging have focused on blocking and possibly reversing the pathological processes associated with the physical deterioration of the brain. However, the positive benefits provided by available therapeutic approaches (most notably, the cholinesterase inhibitors) have been modest to date in AD, and are not approved for earlier stages of memory and cognitive loss such as age-related cognitive decline and MCI.

Cognitive training is another potentially potent therapeutic approach to the problems of age-related cognitive decline, MCI, and AD. This approach typically employs computer- or clinician-guided training to teach subjects cognitive strategies to mitigate their memory loss. Although moderate gains in memory and cognitive abilities have been recorded with cognitive training, the general applicability of this approach has been significantly limited by two factors: 1) Lack of Generalization; and 2) Lack of enduring effect.

Lack of Generalization: Training benefits typically do not generalize beyond the trained skills to other types of cognitive tasks or to other “real-world” behavioral abilities. As a result, effecting significant changes in overall cognitive status would require exhaustive training of all relevant abilities, which is typically infeasible given time constraints on training.

Lack of Enduring Effect: Training benefits generally do not endure for significant periods of time following the end of training. As a result, cognitive training has appeared infeasible given the time available for training sessions, particularly from people who suffer only early cognitive impairments and may still be quite busy with daily activities.

As a result of overall moderate efficacy, lack of generalization, and lack of enduring effect, no cognitive training strategies are broadly applied to the problems of age-related cognitive decline, and to date they have had negligible commercial impacts. The applicants believe that a significantly innovative type of training can be developed that will surmount these challenges and lead to fundamental improvements in the treatment of age-related cognitive decline. This innovation is based on a deep understanding of the science of “brain plasticity” that has emerged from basic research in neuroscience over the past twenty years, which only now through the application of computer technology can be brought out of the laboratory and into the everyday therapeutic treatment.

Thus, improved systems and methods for improving cognition and memory are desired.

### SUMMARY

Various embodiments of a system and method for enhancing cognition and memory in a subject via cognitive training exercises using continuous stimuli, i.e., stimulus streams, are presented. Embodiments of the computer-based exercises or tasks described herein may operate to renormalize and improve the ability of the nervous system to perceive, process, and remember, information presented in a continuous manner. This may be achieved by having subjects perform any of various tasks using stimulus streams under conditions of high engagement/stimulation and under high reward for correct performance in order to encourage renormalization of cognition and memory. In some embodiments, the method may be as follows:

A set (or sets) of stimuli may be provided for presentation to the subject. For example, the stimuli may be stored on a memory medium of the computing device, on a memory medium coupled to the computing device, e.g., over a network, etc. The stimuli preferably include visual stimuli, e.g., geometric shapes with various attributes, although in other embodiments, other types of visual stimulus, e.g., orthographic (textual) may be used, or even different stimulus modes, such as, for example, auditory, symbolic, and so forth. Note that as used herein, a “more difficult stimulus” means that in the context of a cognitive training task, the presentation of the stimulus would result in a lower probability of correct response by the subject.

Exemplary visual stimuli suitable for some embodiments of the invention may include various geometric shapes, or groups of shapes, with specified attributes. Note that the stimuli shown are meant to be exemplary only, and are not intended to limit the stimuli to any particular form, type, category, mode, or appearance. The shapes used in some embodiments of the invention may have any of numerous attributes, including, but not limited to, shape, color, texture (interior pattern), and quantity (i.e., number), among others. In some embodiments, the method may include presenting a plurality of exercises or tasks where different categories, groupings, or complexities of attributes are used.

At least one target attribute may be presented to the subject. The at least one target attribute may be presented visually and/or audibly as desired. In some embodiments, the at least one target attribute may specify one or more of the stimulus attributes of stimuli, e.g., a specified shape, texture, color, or quantity. In further embodiments, the at least one target attribute may specify the absence of an attribute, e.g., the absence of a specified color, e.g., not blue, the absence of a specified shape, e.g., not a square, the absence of a specified texture, e.g., not solid, and so forth, for any of the attributes used. In some embodiments, the at least one target attribute may include a plurality of attributes, such as, for example, blue and round, or red, not square, and nested, among others. Thus, the at least one target attribute may include one attribute, the absence of an attribute, or a combination of multiple attributes or absences of attributes, as desired. Various exemplary tasks are described below illustrating exemplary target attributes, although it should be noted that any types of target attributes may be used as desired, e.g., arbitrary attributes of arbitrary stimuli.

A continuous sequence of stimulus groups from the set of stimuli may be presented to the subject one stimulus group at a time, where each stimulus group includes one or more stimuli. Each stimulus group may be presented for a specified duration, and the stimulus groups in the continuous sequence of stimulus groups may be separated by a specified inter-stimulus-interval (ISI).

Note that the continuous sequence of stimulus groups includes or corresponds to one or more continuous stimulus streams, where the one or more stimuli in each stimulus group are from respective continuous stimulus streams of the one or more continuous stimulus streams. In other words, each stimulus group includes stimuli from respective (one or more) stimulus streams, and the presenting of the sequence of stimulus groups includes presenting the stimulus streams to the subject, with each stimulus stream contributing a respective stimulus for each stimulus group. For example, in an example where three stimulus streams are used, the stimulus group may include a solid blue square, a solid blue circle, and a nested blue triangle, which in this case, would match or correspond with target attributes such as “blue”, “not red”, “not green”, “not open”. Other examples of such stimulus groups are described below.

Each stimulus group in the continuous sequence of stimulus groups may be processed as follows:

The subject may be required to respond to the stimulus group by indicating when the one or more stimuli in a stimulus group correspond to the at least one target attribute In some embodiments, the subject may be required to respond differently when the stimulus group does not match or correspond to the at least one target attribute, or to refrain from indicating when the stimulus group does not correspond to the at least one target attribute. Said another way, in these embodiments, when the stimulus group matches or corresponds to the at least one target attribute, the subject should indicate that this is so. However, when the stimulus group does not match or correspond to the at least one target attribute, in some embodiments, the subject should indicate so with a different response, while in other embodiments, the subject should refrain from responding. As noted above, “corresponding” or “matching” stimuli, refer to stimuli that correspond to the specified at least one target attribute. Thus, for example, in some embodiments where the subject is to respond positively when the stimulus group matches, and negatively when the stimulus group doesn't match, the subject may press a first key, e.g., an up arrow key, when the stimulus group matches or corresponds to the at least one target attribute, and may press a second key, e.g., a down arrow key, when the stimulus group doesn't match. Of course, other keys or GUI controls may be used as desired. In embodiments where the subject is to inhibit responses to non-target stimuli, when the stimulus group doesn't match or correspond to the at least one target attribute, the subject should do nothing.

Thus, in a preferred embodiment, one or more stimulus streams (continuous sequence of stimulus groups) may be presented on a visual display device, e.g., a computer monitor, where the subject may be required to respond when the stimulus group matches or corresponds to the at least one target attribute, e.g., by pressing the spacebar on the keyboard, clicking on a GUI control, etc. As noted above, “corresponding” or “matching” stimuli, refer to stimuli that correspond to the specified target attribute(s).

A determination may be made as to whether the subject responded correctly for the stimulus group. For example, in some embodiments where the subject is to inhibit responses to non-targets, the subject's response to the stimulus group may include one of: a correct response, including: a true positive, where the subject correctly indicates when the stimulus group (i.e., the one or more stimuli in the stimulus group) corresponds to or matches the at least one target attribute, or a true negative, where the subject correctly refrains from indicating when the stimulus group does not correspond to or match the at least one target attribute; or an incorrect response, including: a false negative, where the subject (incorrectly) fails to indicate when the stimulus group corresponds to or matches the at least one target attribute, or a false positive, where the subject incorrectly indicates that the stimulus group corresponds to or matches the at least one target attribute. In other words, in these embodiments, if the stimulus group corresponds to or matches the at least one target attribute, the correct response is to indicate the correspondence or match, and the incorrect response is to fail to indicate the correspondence or match, and if the stimulus group does not correspond to or match the at least one target attribute, the correct response is to refrain from indicating a correspondence or match, and the incorrect response is to (incorrectly) indicate that the stimulus group corresponds to or matches the at least one target attribute.

In some embodiments where the subject is to respond by indicating both targets and non-targets, the subject's response to the stimulus group may include one of: a correct response, including: a true positive, where the subject correctly indicates when the stimulus group (i.e., the one or more stimuli in the stimulus group) corresponds to or matches the at least one target attribute, or a true negative, where the subject correctly indicates when the stimulus group does not correspond to or match the at least one target attribute; or an incorrect response, including: a false negative, where the subject incorrectly indicates that the stimulus group does not correspond to or match the at least one target attribute, or a false positive, where the subject incorrectly indicates that the stimulus group corresponds to or matches the at least one target attribute. In other words, if the stimulus group corresponds to or matches the at least one target attribute, the correct response is to indicate the correspondence or match, and the incorrect response is to (incorrectly) indicate that the stimulus group does not correspond to or match the at least one target attribute, and if the stimulus group does not correspond to or match the at least one target attribute, the correct response is to indicate so, and the incorrect response is to (incorrectly) indicate that the stimulus group corresponds to or matches the at least one target attribute.

In one embodiment, the subject may be required to respond before a next stimulus group is presented, i.e., before the stimulus's ISI has elapsed. This time period between the moment the stimulus group is presented and the onset of the subsequent stimulus group is referred to as the window of response, and may be modified per trial. In other words, the response window is the duration of the stimulus presentation plus the following ISI time.

In some embodiments, an indication, e.g., an audible or visual indication, may be provided to the subject indicating whether the subject responded correctly, i.e., indicating the correctness or incorrectness of the subject's response. In some embodiments, indicating whether the subject responded correctly may include rewarding the subject if a specified level of success is achieved, or penalizing the subject if a specified level of failure is achieved, where the rewarding and penalizing may each include one or more of: auditory feedback, visual feedback, point modification, or change in bonus status. Of course, any types of indication may be used as desired, e.g., tokens, graphical images, animation, audible rewards, e.g., tunes, etc.

The duration and/or the ISI may be adjusted based on the determining using an adaptive procedure. For example, if the subject achieves some specified level of success, the duration and/or ISI may be decreased, thereby increasing the difficulty of the task. Conversely, if the subject has achieved some specified level of failure (or failed to achieve a (possibly different) level of success, the duration and/or ISI may be increased, thereby decreasing the difficulty of the task. In preferred embodiments, the adaptive procedure may comprise a maximum likelihood procedure. For example, the maximum likelihood procedure may be or include a QUEST (quick estimation by sequential testing) threshold procedure, or a ZEST (zippy estimation by sequential testing) threshold procedure, described below, whereby threshold values for the stimulus duration and/or ISI (or more generally, the stimulus intensity) may be determined based on the subject's performance. In preferred embodiments, a continuous performance maximum likelihood procedure may be used, e.g., continuous performance ZEST or continuous performance QUEST. However, it should be noted that in various embodiments, any adaptive procedure may be used as desired.

In some embodiments, adjusting the stimulus duration and/or ISI may include adjusting the stimulus duration and/or ISI to approach and substantially maintain a specified success rate for the subject, e.g., using a single stair maximum continuous performance likelihood procedure, also described below.

In one embodiment, the above presenting the at least one target attribute, presenting the sequence of stimulus groups, and the processing each stimulus group in the sequence of stimulus groups (including the response/determination/adjustment) may compose a session. For each session, an initial value of the duration and/or the ISI and a final value of the duration and/or the ISI may be averaged to determine the initial value of the duration and/or the ISI for the next session. In some embodiments, for each session, reaction times (of the subject) may be averaged for each stimulus group in the continuous sequence of stimulus groups to determine a minimum value for the duration and/or the ISI for the next session, where each reaction time includes a respective delay between the presentation of each stimulus group in which all the stimuli correspond to the at least one target attribute and the subject's response to the stimulus group. In some embodiments, at the end of each session a reward may be presented, e.g., visually and/or audibly.

In some embodiments, the method may further include performing an initial session prior to performing the above method elements, where the initial session includes the above method elements, but where, in processing each stimulus group in the initial session, neither the duration nor the ISI is adjusted.

In one embodiment, each stimulus group presentation and corresponding subject response may compose a trial. The method may further include for each trial, recording one or more of: the at least one target attribute, the stimulus group, whether or not the stimulus group corresponds to the at least one target attribute, the duration, the ISI, the subject's response, the correctness or incorrectness of the subject's response, the reaction time for the trial, or statistical measures for the adaptive procedure, e.g., the continuous performance maximum likelihood procedure, described in more detail below.

The above presenting the at least one target attribute, presenting the sequence of stimulus groups, and the processing each stimulus group in the sequence of stimulus groups (including the response/determination/adjustment) may be repeated one or more times in an iterative manner to improve the cognition of the subject. In other words, the above method elements may be iteratively performed to improve the cognition of the subject, e.g., presenting various stimulus group sequences (stimulus streams), and adjusting the stimulus presentation to increase or decrease the task difficulty based on the subject's responses. In some embodiments, the repeating may be terminated if the subject responds incorrectly a specified number of times consecutively, e.g., 5 times in a row.

In some embodiments, the above method elements may be performed under a specified condition, where the condition specifies one or more aspects or attributes of the presenting the continuous sequence(s) of stimulus groups. Moreover, the method may further include performing the repeating a plurality of times, i.e., iteratively, where each iteration is performed under a respective condition. In other words, not only may multiple continuous sequences of groups of stimuli be presented, but a plurality of such multiple presentations may be performed as well, each under a respective condition.

In one embodiment, each condition may specify one or more of: session length, e.g., length of the continuous sequence of stimulus groups, and/or length of time of said presenting the continuous sequence of stimulus groups, correspondence frequency, e.g., a ratio of stimulus groups in which all the stimuli correspond to the at least one target attribute to stimulus groups in which all the stimuli do not correspond to the at least one target attribute (may be shortened or randomized to test subject's ability to focus attention and recall targets in various frequencies), or target/foil confusability, e.g., a degree to which stimuli that correspond to the at least one target attribute are similar to stimuli that do not correspond. In other embodiments, other attributes may be used as desired.

As noted above, in some embodiments, the conditions may become more difficult as the subject progresses through the exercise. For example, various conditions or types of target attributes may correspond to different progressive levels in the exercise, through which the subject may progress as the exercise or task is performed. Moreover, the method may include various continuous performance exercises or tasks, where different types or categories of stimuli may be used.

In some embodiments, the exercise may include a combination of tasks. For example, in one embodiment, the presenting at least one target attribute, presenting the continuous sequence of stimulus groups, and the requiring, determining, and adjusting may compose a session. The repeating may include two or more of: performing one or more sessions where the one or more stimuli include a plurality of attributes, including two or more of: color, shape, or texture, and where the at least one target attribute includes one or more of: color, shape, texture, absence of a specified color, absence of a specified shape, or absence of a specified texture, among others; and performing one or more sessions where each stimulus group includes a plurality of stimuli, where the at least one target attribute includes one or more relationships of attributes of the plurality of stimuli, each relationship including one or more of: identical, wherein each stimulus of the plurality of stimuli has a common attribute value, or distinct, wherein each stimulus of the plurality of stimuli has a different attribute value, and each of the plurality of stimuli including a plurality of attributes, including one or more of: color, shape, texture, or quantity, absence of a specified color, absence of a specified shape, absence of a specified texture, or absence of a specified quantity, among others.

In some embodiments, the repeating, and/or the performing the repeating a plurality of times, may occur a specified number of times each day, for a specified number of days. In other words, the subject may perform a plurality of sessions each day over a period of days, e.g., for 6 months, to improve cognition.

In some embodiments, the method may also include performing one or more practice sessions, i.e., prior to performing the method elements described above. For example, in some embodiments, one or more practice sessions may be performed prior to the beginning of training to familiarize the subject with the nature and mechanisms of the exercise. In some embodiments, in each practice session, a specified number of trials (e.g., 1) for each of one or more practice conditions may be performed. In some embodiments, the subject may be able to invoke such practice sessions at will during the exercise, e.g., to re-familiarize the subject with the task at hand.

As indicated above, in some embodiments, the duration and/or ISI may be adjusted using an adaptive procedure. For example, in some embodiments, the duration and/or ISI may be adjusted using a maximum likelihood procedure. Such procedures may be used to modify or set an adjustable attribute (or combination of attributes) of a presented stimulus, whereby trials in the task or exercise may be made more or less difficult. Such an adjustable parameter is generally referred to as a stimulus intensity, and the maximum likelihood procedure is used to determine a stimulus threshold, which is the value of the stimulus intensity at which the subject achieves a specified level of success, e.g., 0.9, corresponding to a 90% success rate. There are various approaches whereby such thresholds may be assessed or determined, such as, for example, the well known QUEST (Quick Estimation by Sequential Testing) threshold method, which is an adaptive psychometric procedure for use in psychophysical experiments, or a related method, referred to as the ZEST (Zippy Estimation by Sequential Testing) procedure or method, among others.

Exercise based threshold determination may be designed to assess a subject's threshold with respect to stimuli on a given exercise, and can be used to adjust stimulus presentation to achieve and maintain a desired success rate for the subject, e.g., with respect to a particular exercise, task, and/or condition. In preferred embodiments of the exercises and tasks described herein, the stimulus intensity is the duration and/or ISI of the presented stimuli. In other words, the progressions (successive modifications or adjustments of presentation parameters, e.g., duration and/or ISI) in the exercise may be calculated using adaptive procedure, e.g., a maximum likelihood procedure, e.g., the ZEST procedure. For example, for each trial, a likelihood function may be calculated (based on the subject's response) to determine the next best guess of the true threshold. This estimate may be used (and possibly displayed) in the next trial. Based on the trial outcome and all previous trials the next best guess may be calculated and used in the presentation of stimuli for the next trial (and possibly displayed). As the probability function narrows and the standard deviation decreases, the estimate of true threshold approaches the true value—e.g., achieving a “good enough” threshold value. Note that the ZEST procedure is a modification of the QUEST procedure—the ZEST procedure uses the mean while the QUEST procedure uses the mode. In preferred embodiments, when training, a single stair ZEST procedure may be used, e.g., with a threshold level of 85%, although other values may be used as desired.

In preferred embodiments, the maximum likelihood procedure may be a continuous performance maximum likelihood procedure (as opposed to a discrete performance maximum likelihood procedure), such as a continuous performance ZEST procedure. One such a procedure, referred to as a continuous performance task (CPT), is described below.

The Continuous Performance Task (CPT) is a neuropsychological task or exercise that consists of a series of stimulus groups presented one after another. The subject may be instructed to attend to a particular stimulus or a category of stimuli and respond to the presented stimulus group. The initial duration of CPT, e.g., the initial value of the presentation time for the stimulus groups, may differ from task to task.

In preferred embodiments, there are four possible response outcomes: true positive, true negative, false positive and false negative, defined as follows:

Correct responses:
True positive: hit or correct response to a correct target, or
True negative: no response to a non-target (or correct response to a non-target).

Incorrect responses:
False negative: miss or no response to a correct target, or
False positive: response to a non-target (or incorrect response to a non-target).

Continuous Performance Tasks have traditionally been conducted where the stimulus Onset Asynchrony (SOA) time, e.g., the duration plus the ISI, is fixed throughout a session or is only altered after a block of stimuli is presented. Because the appropriate SOA time may differ greatly depending on: 1) the nature of the task; 2) the type of stimulus set used; and 3) the ability of the participant, a progression algorithm that optimizes the training experience is desired, especially when the training of speed of processing is crucial. For example, if the SOA time is too long, the task will not challenge the participants and thus not engage them. If the SOA time is too short, participants may find the task frustrating and the task may lack training value. Thus, a new SOA time progression scheme is presented herein that optimizes training experience by changing the SOA from trial to trial using an adaptive procedure, e.g., based on a maximum likelihood method. This scheme is used in preferred embodiments of the continuous performance cognitive training exercises described above.

In one embodiment, the progression scheme employed is based on the ZEST (Zippy Estimation by Sequential Testing) Bayesian adaptive estimator method, which is a probabilistic procedure where a prior estimate value of a variable is updated sequentially via a likelihood function that contains all the previous trials' information, yielding a posterior estimate value that encompasses all the data generated so far from the initial assumptions to all the user's responses. In other embodiments, other adaptive procedures may be used, e.g., QUEST.

Other features and advantages of the present invention will become apparent upon study of the remaining portions of the specification and drawings.

### BRIEF DESCRIPTION OF THE DRAWINGS

FIG. 1 is a block diagram of a computer system for executing a program according to some embodiments of the present invention;

FIG. 2 is a block diagram of a computer network for executing a program according to some embodiments of the present invention;

FIG. 3 is a high-level flowchart of one embodiment of a method for cognitive training using stimulus streams with target attributes, according to one embodiment;

FIGS. 4A-4D illustrate exemplary visual stimuli grouped by attribute, according to one embodiment;

FIG. 5 illustrates various examples of visual stimuli that match target attributes, according to one embodiment;

FIG. 6A illustrates an exemplary graphical user interface presenting task instructions and a target attribute, according to one embodiment;

FIG. 6C illustrates an exemplary graphical user interface (GUI) presenting task instructions and a target attribute, according to one embodiment;

FIG. 6B illustrates an exemplary screenshot of a GUI indicating a correct response to a trial with respect to the target attribute of FIG. 6A, according to one embodiment;

FIG. 6C illustrates an exemplary screenshot of a GUI indicating an incorrect response to a trial with respect to the target attribute of FIG. 6A, according to one embodiment;

FIG. 7 illustrates various examples of visual stimuli that match target attribute relationships, according to one embodiment;

FIG. 7 illustrates an exemplary response inhibition task using auditory and visual directions, according to one embodiment;

FIG. 8A illustrates an exemplary screenshot of a GUI presenting task instructions and a target attribute relationship, according to one embodiment;

FIG. 8B illustrates an exemplary screenshot of a GUI presenting a stimulus group based on three concurrent stimulus streams, according to one embodiment;

FIG. 9A illustrates an exemplary screenshot of a graphical user interface indicating a correct response to a trial, according to one embodiment;

FIG. 9B illustrates an exemplary screenshot of a graphical user interface indicating an incorrect response to a trial, according to one embodiment;

FIG. 10 illustrates an exemplary psychometric function based on a cumulative Gaussian distribution for a detection (yes/no) task with a 5% lapsing rate, and a target performance rate of 85%, according to one embodiment;

FIG. 11 illustrates an exemplary probability density function with initial intensity estimate x=5, according to one embodiment;

FIG. 12 illustrates exemplary likelihood functions with T=5 and an 85% correct rate, according to one embodiment;

FIG. 13 illustrates an exemplary prior probability density function (P.D.F.) superimposed with exemplary likelihood functions, according to one embodiment; and

FIG. 14 illustrates an exemplary posterior P.D.F. with Yes/No response, according to one embodiment.

### DETAILED DESCRIPTION

Below are described various embodiments of a system and method for continuous performance cognitive training using stimulus streams.

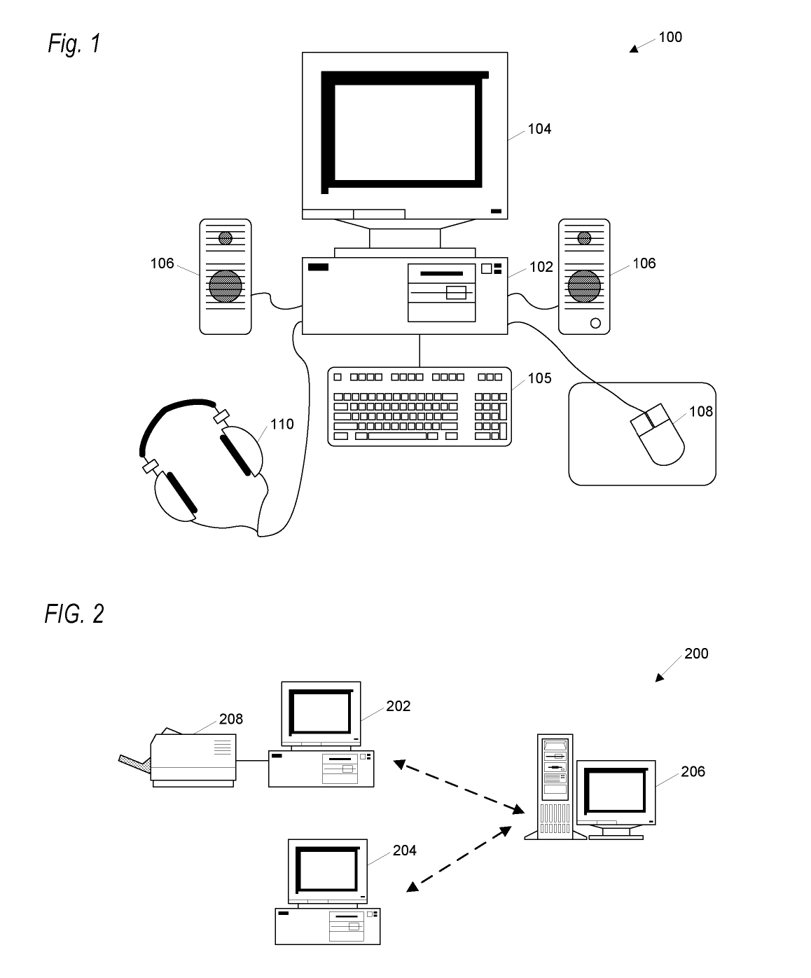

Referring to FIG. 1, a computer system 100 is shown for executing a computer program to train, or retrain an individual according to the present invention to enhance cognition, where the term “cognition” refers to the speed, accuracy and reliability of processing of information, and attention and/or memory, and where the term “attention” refers to the facilitation of a target and/or suppression of a non-target, e.g., over a given spatial extent, object-specific area, or time window, e.g., with respect to one or more stimulus streams, such as in a continuous performance exercise. As shown, in this embodiment, the computer system 100 contains a computer 102, having a CPU, memory, hard disk and CD ROM drive (not shown), attached to a monitor 104. The monitor 104 provides visual prompting and feedback to the subject during execution of the computer program. Attached to the computer 102 are a keyboard 105, speakers 106, a mouse 108, and headphones 110. In some embodiments, the speakers 106 and the headphones 110 may provide auditory prompting, stimuli, and feedback to the subject during execution of the computer program. The mouse 108 allows the subject to navigate through the computer program, and to select particular responses after visual or auditory prompting by the computer program. The keyboard 105 allows an instructor to enter alphanumeric information about the subject into the computer 102, and/or response by the subject. Although a number of different computer platforms are applicable to the present invention, embodiments of the present invention execute on either IBM compatible computers or Macintosh computers, or similarly configured computing devices such as set top boxes, PDA's, gaming consoles, etc.

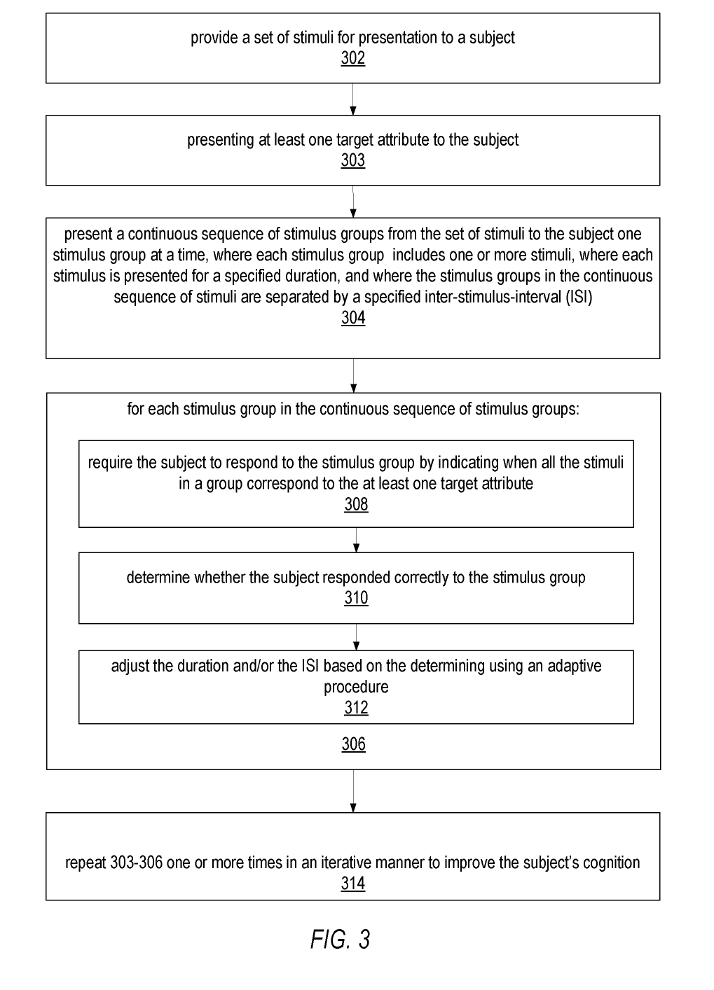

Now referring to FIG. 2, a computer network 200 is shown. The computer network 200 contains computers 202, 204, similar to that described above with reference to FIG. 1, connected to a server 206. The connection between the computers 202, 204 and the server 206 can be made via a local area network (LAN), a wide area network (WAN), or via modem connections, directly or through the Internet. A printer 208 is shown connected to the computer 202 to illustrate that a subject can print out reports associated with the computer program of the present invention. The computer network 200 allows information such as test scores, game statistics, and other subject information to flow from a subject's computer 202, 204 to a server 206. An administrator can review the information and can then download configuration and control information pertaining to a particular subject, back to the subject's computer 202, 204.

Embodiments of the computer-based exercises or tasks described herein may operate to renormalize and improve the ability of the nervous system to perceive, process, and remember, information via continuous performance using stimulus streams. This may be achieved by having subjects perform any of various tasks using stimuli under conditions of high engagement/stimulation and under high reward for correct performance in order to encourage renormalization of cognition, and memory.

More specifically, embodiments of the systems and methods presented herein may be used to train subjects to maintain attention and vigilance on one or more streams of information, e.g., stimulus streams. In a preferred embodiment, one or more visual stimulus streams, e.g., streams of geometric shapes, may be used, although in other embodiments, other stimulus modes may be used, such as, for example, auditory, orthographic (textual), and so forth. As the stimulus stream or streams are presented, the subject is to respond positively when the stimulus matches or corresponds to a specified attribute or attribute relationship, while inhibiting positive response when non-matches are presented. As subject's progress through the exercise, the presentation duration and/or the inter-stimulus-interval (ISI) of the stimuli may be shortened to require quicker and more accurate responses from the subject. Additionally, over the course of the exercise, stimulus streams may be presented under a variety of conditions, described below, where the conditions may become more difficult as the subject progresses through the exercise.

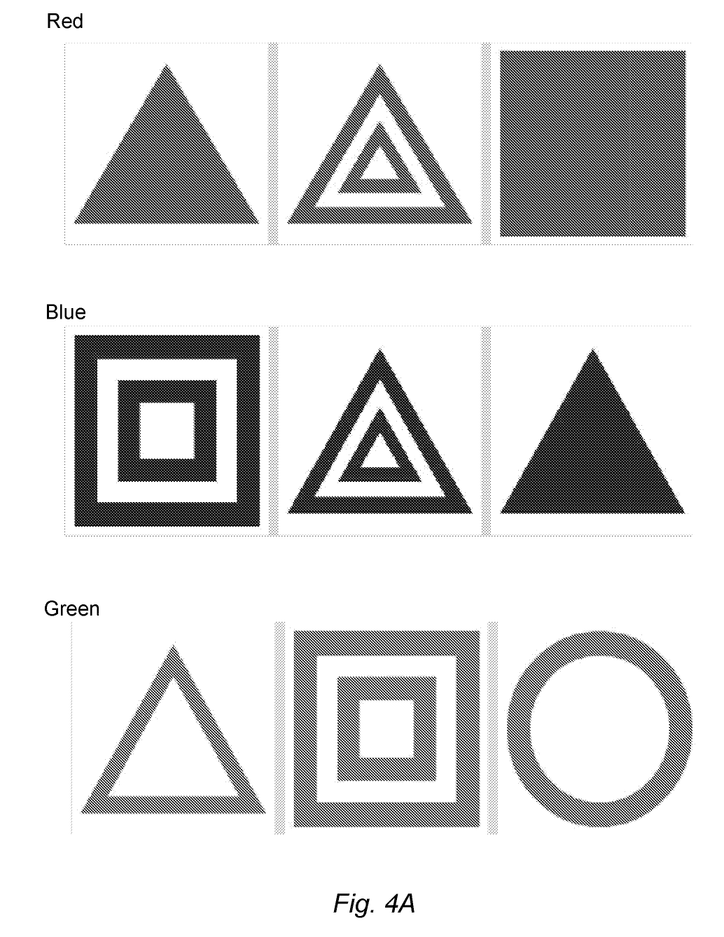

FIG. 3—Flowchart of a Method for Cognitive Training Using Continuous Performance

FIG. 3 is a high-level flowchart of one embodiment of a method for cognitive training using continuous stimuli, i.e., stimulus streams. More specifically, the method utilizes a computing device to present one or more stimulus streams, and to record responses from the subject. A primary goal of this method is to challenge subjects to engage in a continuous task in which the participant must make quick analytical and organizational decisions across streams of information on the basis of one or more criteria. The subject is to respond positively when presented with the desired target, and to respond negatively or refrain from indicating when presented with non-target stimuli. As the subject progresses through the exercise, the presentation of the stimuli may be shortened to require quicker and more accurate response from the subject, specifically, via an adaptive procedure, such as, for example, a continuous performance maximum likelihood procedure. Successful completion of the task may improve the concentration and focus system as well as elevating the speed of processing.

The method may be used in the context of any of a variety of cognitive training exercises using continuous performance with stimulus streams, examples of which are described below. It should be noted that in various embodiments, some of the method elements may be performed concurrently, in a different order than shown, or may be omitted. Additional method elements may also be performed as desired. As shown, the method may be performed as follows:

In 302, a set (or sets) of stimuli may be provided for presentation to the subject. For example, the stimuli may be stored on a memory medium of the computing device, on a memory medium coupled to the computing device, e.g., over a network, etc. The stimuli preferably include visual stimuli, e.g., geometric shapes with various attributes, although in other embodiments, other types of visual stimulus, e.g., orthographic (textual) may be used, or even different stimulus modes, such as, for example, auditory, symbolic, and so forth. Note that as used herein, a “more difficult stimulus” means that in the context of a cognitive training task, the presentation of the stimulus would result in a lower probability of correct response by the subject.

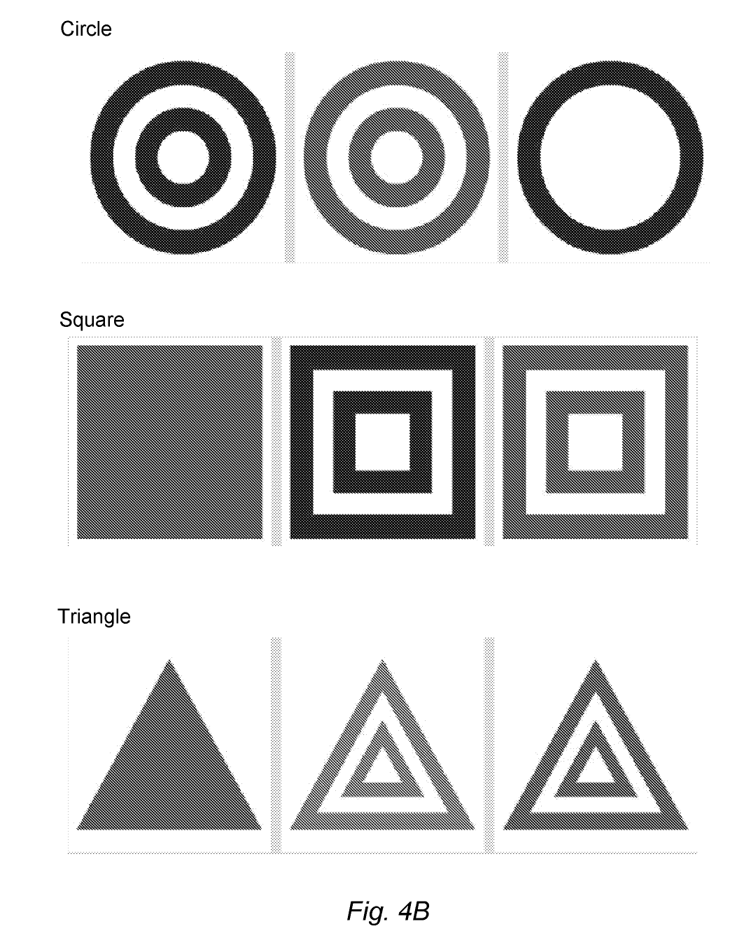

FIGS. 4A-4D illustrate exemplary visual stimuli suitable for some embodiments of the invention, specifically, various geometric shapes, or groups of shapes, with specified attributes. Note that the stimuli shown are meant to be exemplary only, and are not intended to limit the stimuli to any particular form, type, category, mode, or appearance. The shapes used in some embodiments of the invention may have any of numerous attributes, including, but not limited to, shape, color, texture (interior pattern), and quantity (i.e., number), among others. As will be discussed below in more detail, in some embodiments, the method may include presenting a plurality of exercises or tasks where different categories, groupings, or complexities of attributes are used.

FIG. 4A illustrates various different shapes with a specified color attribute, where, as may be seen, the top row shows red shapes, the middle row shows blue shapes, and the bottom row shows green shapes. Note that within each row the particular shapes and textures may differ, but the colors are the same, thus each shape (stimulus) in a row would properly correspond to or match that specified color as a target attribute.

FIG. 4B illustrates various different shapes with a specified shape attribute, where, as may be seen, the top row shows circles, the middle row shows squares, and the bottom row shows triangles. Note that within each row the particular colors and textures may differ, but the shapes are the same, thus each shape (stimulus) in a row would properly correspond to or match that specified shape as a target attribute.

FIG. 4C illustrates various different shapes with a specified texture attribute, where, as may be seen, the top row shows open (i.e., hollow) shapes, the middle row shows solid shapes, and the bottom row shows nested shapes. Note that within each row the particular shapes and colors may differ, but the textures are the same, thus each shape (stimulus) in a row would properly correspond to or match that specified texture as a target attribute.

Finally, FIG. 4D illustrates various different shapes with a specified quantity or number attribute, where, as may be seen, the top row shows single shapes, the middle row shows pairs of shapes, and the bottom row shows triplets of shapes. Note that within each row the particular shapes, colors, and textures may differ, but the quantities of each example stimulus are the same, thus each stimulus in a row would properly correspond to or match that specified quantity as a target attribute.

In 303, at least one target attribute may be presented to the subject. The at least one target attribute may be presented visually and/or audibly as desired. In some embodiments, the at least one target attribute may specify one or more of the stimulus attributes of stimuli, e.g., a specified shape, texture, color, or quantity. In further embodiments, the at least one target attribute may specify the absence of an attribute, e.g., the absence of a specified color, e.g., not blue, the absence of a specified shape, e.g., not a square, the absence of a specified texture, e.g., not solid, and so forth, for any of the attributes used. In some embodiments, the at least one target attribute may include a plurality of attributes, such as, for example, blue and round, or red, not square, and nested, among others. Thus, the at least one target attribute may include one attribute, the absence of an attribute, or a combination of multiple attributes or absences of attributes, as desired. Various exemplary tasks are described below illustrating exemplary target attributes, although it should be noted that any types of target attributes may be used as desired, e.g., arbitrary attributes of arbitrary stimuli.

In 304, a continuous sequence of stimulus groups from the set of stimuli may be presented to the subject one stimulus group at a time, where each stimulus group includes one or more stimuli. Each stimulus group may be presented for a specified duration, and the stimulus groups in the continuous sequence of stimulus groups may be separated by a specified inter-stimulus-interval (ISI).

Note that the continuous sequence of stimulus groups includes or corresponds to one or more continuous stimulus streams, where the one or more stimuli in each stimulus group are from respective continuous stimulus streams of the one or more continuous stimulus streams. In other words, each stimulus group includes stimuli from respective (one or more) stimulus streams, and the presenting of the sequence of stimulus groups includes presenting the stimulus streams to the subject, with each stimulus stream contributing a respective stimulus for each stimulus group. For example, in an example where three stimulus streams are used, the stimulus group may include a solid blue square, a solid blue circle, and a nested blue triangle, which in this case, would match or correspond with target attributes such as “blue”, “not red”, “not green”, “not open”. Other examples of such stimulus groups are described below.

As FIG. 3 indicates, in 306, each stimulus group in the continuous sequence of stimulus groups may be processed as follows (308-312):

In 308, the subject may be required to respond to the stimulus group by indicating when the one or more stimuli in a stimulus group correspond to the at least one target attribute In some embodiments, the subject may be required to respond differently when the stimulus group does not match or correspond to the at least one target attribute, or to refrain from indicating when the stimulus group does not correspond to the at least one target attribute. Said another way, in these embodiments, when the stimulus group matches or corresponds to the at least one target attribute, the subject should indicate that this is so. However, when the stimulus group does not match or correspond to the at least one target attribute, in some embodiments, the subject should indicate so with a different response, while in other embodiments, the subject should refrain from responding. As noted above, “corresponding” or “matching” stimuli, refer to stimuli that correspond to the specified at least one target attribute. Thus, for example, in some embodiments where the subject is to respond positively when the stimulus group matches, and negatively when the stimulus group doesn't match, the subject may press a first key, e.g., an up arrow key, when the stimulus group matches or corresponds to the at least one target attribute, and may press a second key, e.g., a down arrow key, when the stimulus group doesn't match. Of course, other keys or GUI controls may be used as desired. In embodiments where the subject is to inhibit responses to non-target stimuli, when the stimulus group doesn't match or correspond to the at least one target attribute, the subject should do nothing.

Thus, in a preferred embodiment, one or more stimulus streams (continuous sequence of stimulus groups) may be presented on a visual display device, e.g., a computer monitor, where the subject may be required to respond when the stimulus group matches or corresponds to the at least one target attribute, e.g., by pressing the spacebar on the keyboard, clicking on a GUI control, etc. As noted above, “corresponding” or “matching” stimuli, refer to stimuli that correspond to the specified target attribute(s).

In 310, a determination may be made as to whether the subject responded correctly for the stimulus group. For example, in some embodiments where the subject is to inhibit responses to non-targets, the subject's response to the stimulus group may include one of: a correct response, including: a true positive, where the subject correctly indicates when the stimulus group (i.e., the one or more stimuli in the stimulus group) corresponds to or matches the at least one target attribute, or a true negative, where the subject correctly refrains from indicating when the stimulus group does not correspond to or match the at least one target attribute; or an incorrect response, including: a false negative, where the subject (incorrectly) fails to indicate when the stimulus group corresponds to or matches the at least one target attribute, or a false positive, where the subject incorrectly indicates that the stimulus group corresponds to or matches the at least one target attribute. In other words, in these embodiments, if the stimulus group corresponds to or matches the at least one target attribute, the correct response is to indicate the correspondence or match, and the incorrect response is to fail to indicate the correspondence or match, and if the stimulus group does not correspond to or match the at least one target attribute, the correct response is to refrain from indicating a correspondence or match, and the incorrect response is to (incorrectly) indicate that the stimulus group corresponds to or matches the at least one target attribute.

In some embodiments where the subject is to respond by indicating both targets and non-targets, the subject's response to the stimulus group may include one of: a correct response, including: a true positive, where the subject correctly indicates when the stimulus group (i.e., the one or more stimuli in the stimulus group) corresponds to or matches the at least one target attribute, or a true negative, where the subject correctly indicates when the stimulus group does not correspond to or match the at least one target attribute; or an incorrect response, including: a false negative, where the subject incorrectly indicates that the stimulus group does not correspond to or match the at least one target attribute, or a false positive, where the subject incorrectly indicates that the stimulus group corresponds to or matches the at least one target attribute. In other words, if the stimulus group corresponds to or matches the at least one target attribute, the correct response is to indicate the correspondence or match, and the incorrect response is to (incorrectly) indicate that the stimulus group does not correspond to or match the at least one target attribute, and if the stimulus group does not correspond to or match the at least one target attribute, the correct response is to indicate so, and the incorrect response is to (incorrectly) indicate that the stimulus group corresponds to or matches the at least one target attribute.

In one embodiment, the subject may be required to respond before a next stimulus group is presented, i.e., before the stimulus's ISI has elapsed. This time period between the moment the stimulus group is presented and the onset of the subsequent stimulus group is referred to as the window of response, and may be modified per trial. In other words, the response window is the duration of the stimulus presentation plus the following ISI time.

In some embodiments, an indication, e.g., an audible or visual indication, may be provided to the subject indicating whether the subject responded correctly, i.e., indicating the correctness or incorrectness of the subject's response. For example, in one embodiment, a “ding” or a “thunk” (or corresponding equivalents) may be played to indicate correctness or incorrectness, respectively. In some embodiments, indicating whether the subject responded correctly may include rewarding the subject if a specified level of success is achieved, or penalizing the subject if a specified level of failure is achieved, where the rewarding and penalizing may each include one or more of: auditory feedback, visual feedback, point modification, or change in bonus status. Of course, any types of indication may be used as desired, e.g., tokens, graphical images, animation, audible rewards, e.g., tunes, etc.

In 312, the duration and/or the ISI may be adjusted based on the determining of 308, using an adaptive procedure. For example, if the subject achieves some specified level of success, the duration and/or ISI may be decreased, thereby increasing the difficulty of the task. Conversely, if the subject has achieved some specified level of failure (or failed to achieve a (possibly different) level of success, the duration and/or ISI may be increased, thereby decreasing the difficulty of the task. In preferred embodiments, the adaptive procedure may comprise a maximum likelihood procedure. For example, the maximum likelihood procedure may be or include a QUEST (quick estimation by sequential testing) threshold procedure, or a ZEST (zippy estimation by sequential testing) threshold procedure, described below, whereby threshold values for the stimulus duration and/or ISI (or more generally, the stimulus intensity) may be determined based on the subject's performance. In preferred embodiments, a continuous performance maximum likelihood procedure may be used, e.g., continuous performance ZEST or continuous performance QUEST. However, it should be noted that in various embodiments, any adaptive procedure may be used as desired.

In some embodiments, adjusting the stimulus duration and/or ISI may include adjusting the stimulus duration and/or ISI to approach and substantially maintain a specified success rate for the subject, e.g., using a single stair maximum continuous performance likelihood procedure, also described below.

In one embodiment, the method elements 303-306 (including 308-312) may compose a session. In other words, the above presenting at least one target attribute (303), presenting the continuous sequence of stimulus groups (304), and processing each stimulus group (306), including the requiring (308), determining (310), and adjusting (312) for each stimulus group may compose a session. For each session, an initial value of the duration and/or the ISI and a final value of the duration and/or the ISI may be averaged to determine the initial value of the duration and/or the ISI for the next session. In some embodiments, for each session, reaction times (of the subject) may be averaged for each stimulus group in the continuous sequence of stimulus groups to determine a minimum value for the duration and/or the ISI for the next session, where each reaction time includes a respective delay between the presentation of each stimulus group in which all the stimuli correspond to the at least one target attribute and the subject's response to the stimulus group. In some embodiments, at the end of each session a reward may be presented, e.g., visually and/or audibly.

In some embodiments, the method may further include performing an initial session prior to performing 303-306, where the initial session includes 303-306, but where, in performing 306 in the initial session, neither the duration nor the ISI is adjusted.

In one embodiment, each stimulus group presentation and corresponding subject response may compose a trial. The method may further include for each trial, recording one or more of: the at least one target attribute, the stimulus group, whether or not the stimulus group corresponds to the at least one target attribute, the duration, the ISI, the subject's response, the correctness or incorrectness of the subject's response, the reaction time for the trial, or statistical measures for the adaptive procedure, e.g., the continuous performance maximum likelihood procedure, described in more detail below.

In 314, the method elements 303-306 (including 308-312) may be repeated one or more times in an iterative manner to improve the cognition of the subject. In other words, the above presenting at least one target attribute (303), presenting the continuous sequence of stimulus groups (304), and processing each stimulus group (306), including the requiring (308), determining (310), and adjusting (312) for each stimulus group, may be iteratively performed to improve the cognition of the subject, e.g., presenting various stimulus group sequences (stimulus streams), and adjusting the stimulus presentation to increase or decrease the task difficulty based on the subject's responses. In some embodiments, the repeating of 314 may be terminated if the subject responds incorrectly a specified number of times consecutively, e.g., 5 times in a row.

In some embodiments, the above method elements 303-306 may be performed under a specified condition, where the condition specifies one or more aspects or attributes of the presenting the continuous sequence(s) of stimulus groups. Moreover, the method may further include performing the repeating of 314 a plurality of times, i.e., iteratively, where each iteration is performed under a respective condition. In other words, not only may multiple continuous sequences of groups of stimuli be presented (the repeating of 314), but a plurality of such multiple presentations may be performed as well, each under a respective condition.

In one embodiment, each condition may specify one or more of: session length, e.g., length of the continuous sequence of stimulus groups, and/or length of time of said presenting the continuous sequence of stimulus groups, correspondence frequency, e.g., a ratio of stimulus groups in which all the stimuli correspond to the at least one target attribute to stimulus groups in which all the stimuli do not correspond to the at least one target attribute (may be shortened or randomized to test subject's ability to focus attention and recall targets in various frequencies), or target/foil confusability, e.g., a degree to which stimuli that correspond to the at least one target attribute are similar to stimuli that do not correspond. In other embodiments, other attributes may be used as desired.

As noted above, in some embodiments, the conditions may become more difficult as the subject progresses through the exercise. For example, various conditions or types of target attributes may correspond to different progressive levels in the exercise, through which the subject may progress as the exercise or task is performed. Examples of level criteria are provided below.

Moreover, the method may include various continuous performance exercises or tasks, where different types or categories of stimuli may be used, exemplary embodiments of which are described below.

### Exemplary Embodiments of Continuous Performance Convergence Divergence Tasks

The following describes various exemplary embodiments of continuous performance convergence divergence tasks, following the method of FIG. 3, described above, where once the session begins, one or more continuous streams of stimuli are presented, e.g., in the form of a sequence of stimulus groups, each stimulus group composed of one or more images (e.g., figures or shapes), and where the subject is required to respond by pressing the spacebar on the keyboard (or other indication means) when the stimulus matches the criteria (the at least one target attribute) described at the beginning of the session.

Note that the tasks described below are exemplary only, and are not intended to limit the exercise to any particular set of tasks or types of stimuli.

Task 1: Attributes of a Single Stream of Stimuli

In one exemplary task (referred to herein as Task 1 for convenience), a description of the at least one target attribute may appear on the screen, and the subject may press a key on the keyboard to begin. Single stimuli (e.g., figures or shapes) may appear in a continuous stream on the screen. The task is to press the spacebar every time a stimulus appears that matches the attribute or attributes described. In this exemplary task, the attributes include color, shape, texture, and the absence or negative of any of these attributes. One exemplary target attribute may be: “red and not square”.

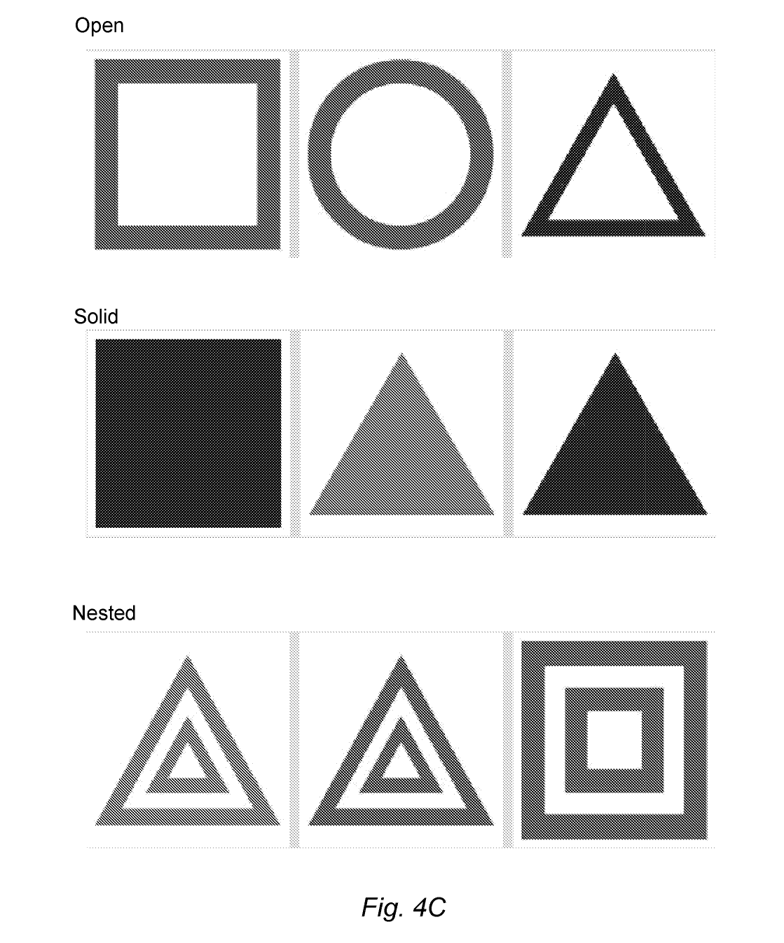

FIG. 5 illustrates example visual stimuli that correspond to various attributes. For example, the top row of stimuli includes three examples where each presented shape corresponds to or matches the attribute “blue”. Thus, in this case, for each of these examples, if the target attribute were “blue” or equivalent (e.g., “figure: blue”), the subject should indicate correspondence, i.e., a match. Note that each of the stimuli may also be considered to correspond to or match other attributes as well, e.g., “not green”. As FIG. 5 also indicates, the bottom row of stimuli includes three examples where each presented shape corresponds to or matches two attributes: “solid” and “not a circle”, specifically, a solid blue triangle, a solid green triangle, and a solid square, each of which corresponds to both of these attributes. As with the top row of example, stimuli, these stimuli also match various other attributes, e.g., “not open” and “not nested”, among others.

In one embodiment, the stimuli may be restricted in form and/or content to some specified set of stimuli. For example, the at least one target attribute may be restricted to a subset of shape, color, texture, and quantity or number, as with exemplary Task 1 (where quantity is not a considered attribute), and each stimulus in the stimulus stream may be restricted to a specified set of shapes or figures, each with a specified subset of attributes. Note however, that in different embodiments, any sets of shapes and attributes (or numbers of streams and numbers of attributes) may be used as desired.

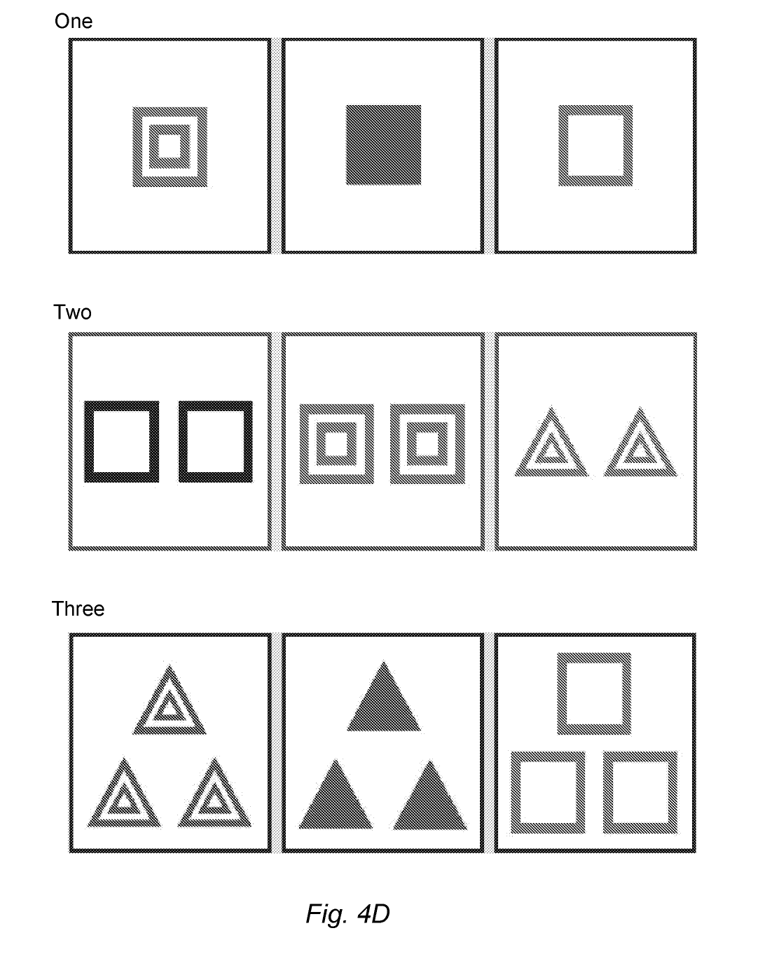

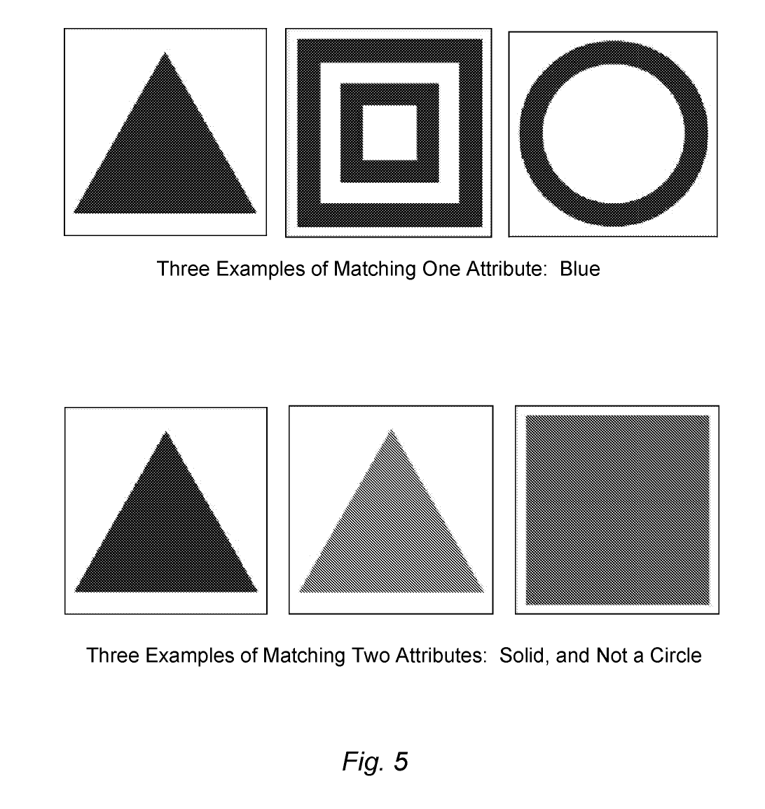

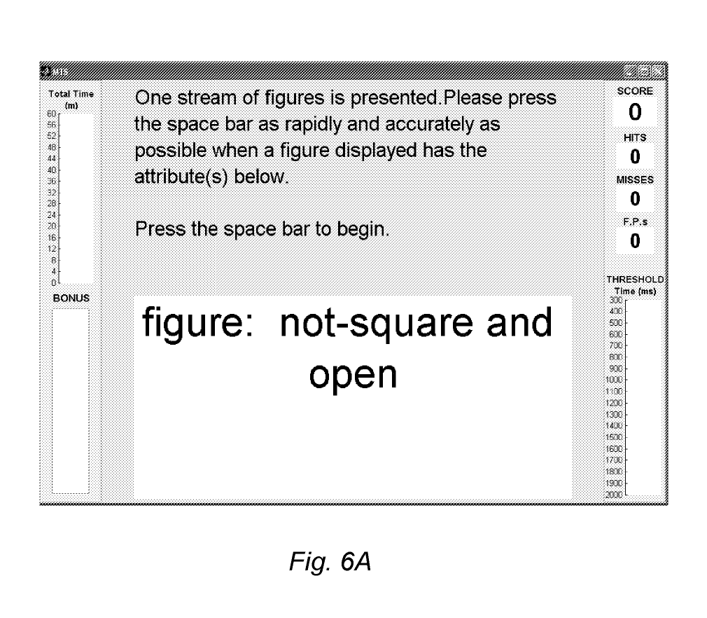

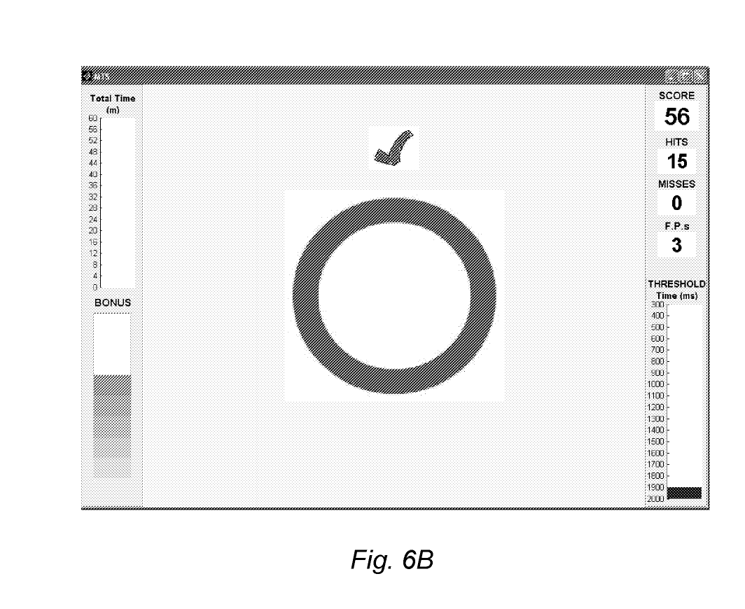

In preferred embodiments, the exercises or tasks described herein are performed via a graphical user interface (GUI), examples of which are described below. In one embodiment, instructions may be presented to the user prior to 303, e.g., explaining to the subject what to expect, and what is expected of the subject in the exercise or task. FIG. 6A is a screenshot of an exemplary GUI for the example Task 1, according to one embodiment. As FIG. 6A shows, in this embodiment, instructions are presented indicating that in this task or exercise, the subject is to press the space bar as quickly and accurately as possible when a stimulus (e.g., a figure) is displayed that matches or corresponds to the at least one target attribute. As may be seen, the GUI also displays the at least one target attribute, in this case: “not-square and open”, meaning that the subject is to respond positively (hitting the space bar) whenever a stimulus (e.g., a figure) is shown that is not a square and that is open (i.e., hollow).

FIG. 6B is a screenshot of an exemplary GUI according to the example Task 1 described above, where the stimulus presented (in a visual or display field of the GUI) is a red open circle, which clearly corresponds to or matches the target attribute “not-square and open”. As may be seen, the subject has apparently responded correctly, as a “check” is displayed, indicating a correct response. FIG. 6C is a screenshot of an exemplary GUI according to the example Task 1 described above, where the stimulus presented is a red solid square, which clearly does not correspond to or match the target attribute “not-square and open”. As may be seen, the subject has apparently responded incorrectly, as an “X” is displayed, indicating an incorrect response.

Summarizing some aspects of the exemplary task described above, the one or more stimuli may include a plurality of attributes, including two or more of: color, shape, or texture, and where the at least one target attribute includes one or more of: color, shape, texture, absence of a specified color, absence of a specified shape, or absence of a specified texture. Of course, other attributes and/or target attributes may be used as desired.

As noted above, in some embodiments, the exercise or task may include various levels through which the subject may progress as the exercise or task is performed. One example of level criteria for Task 1 may include:

Level 1: single attribute;

Level 2: two attributes; and

Level 3: three attributes.

It should be noted, however, that any other level criteria may be used as desired, the above being exemplary only.

Thus, in various embodiments, the method may include one or more tasks or exercises where at least one target attribute is presented, after which a single stream of stimuli (i.e., where the stimulus groups include one element each) is presented, and the user indicates when a stimulus matches or corresponds to the specified target attribute(s). Note, however, that in other embodiments, multiple stimulus streams may be used (i.e., where the stimulus groups include more than one element each). Note also that in embodiments where “quantity” is a possible attribute, each stimulus in the group may include one or more elements, e.g., one square, three triangles, and so forth. Examples of such stimuli are described below with respect to Task 2.

Task 2: Relationship of Attributes of Multiple Streams of Stimuli

In another exemplary task (referred to herein as Task 2 for convenience), the at least one target attribute may specify a relationship of one or more attributes across the stimuli in each stimulus group in a continuous sequence of stimulus groups, where each stimulus group includes a plurality of stimuli. As with Task 1, a description of the at least one target attribute may appear on the screen, and the subject may press a key on the keyboard to begin. Stimulus groups (e.g., figures or shapes) may appear in a continuous stream on the screen, where, as noted above, each stimulus in the group corresponds to a respective stimulus stream. The task is to press the spacebar every time a stimulus group appears that matches the attribute or attributes described. In this exemplary task, the at least one target attribute includes a relationship, e.g., “identical” or “distinct” among one or more stimulus attributes, e.g., color, shape, texture, quantity, and the absence or negative of any of these attributes. Example target attributes include: “identical color”, meaning that all the stimuli in the stimulus group have the same color, and “identical shape and distinct quantity”, meaning that all the stimuli in the stimulus group have the same shape, but each stimulus in the group has a different quantity or number of elements, and so forth.

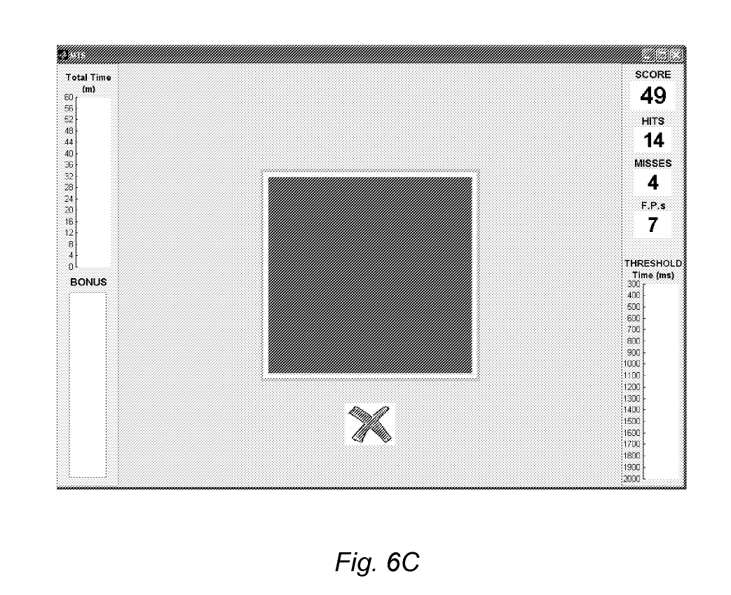

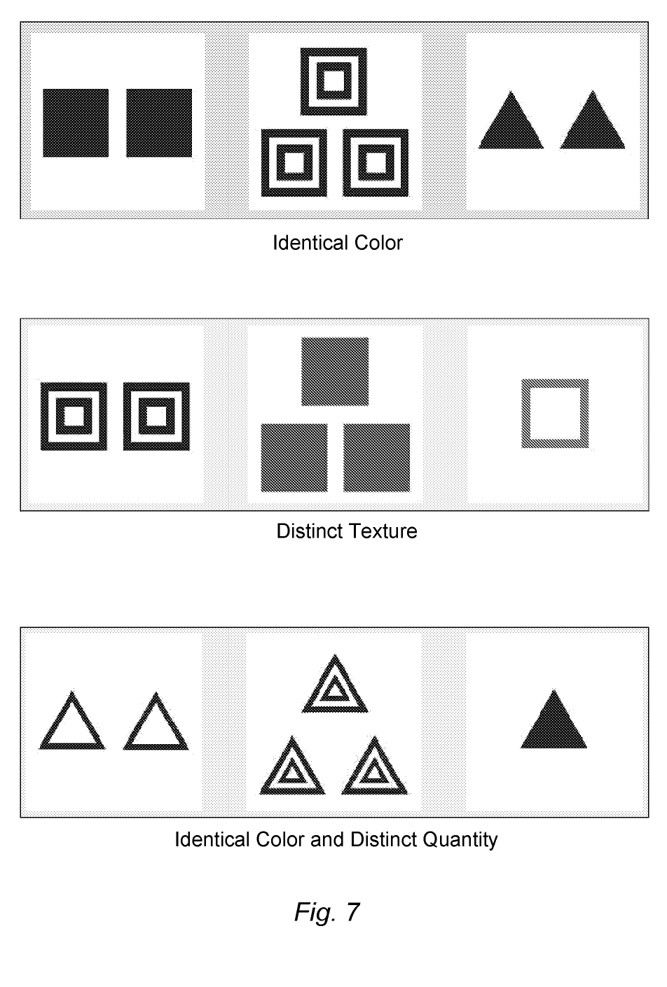

FIG. 7 illustrates example visual stimuli that correspond to or match various attributes, including such relationship attributes. Note that in the examples of FIG. 7, each row illustrates a respective stimulus group, each group including stimuli from three stimulus streams. For example, the top row of stimuli includes an example where each presented shape corresponds to or matches the attribute “identical color”, e.g., a pair of solid blue squares, a triplet of nested blue squares, and a pair of solid blue triangles.

As FIG. 7 also indicates, the middle row of stimuli includes an example where each presented stimulus corresponds to or matches the attribute: “distinct texture”, specifically, a pair of nested blue squares, a triplet of solid red squares, and a single solid green square, each of which corresponds to this attribute. Note that these stimuli may also match various other attributes, e.g., “distinct quantity” and “identical shape”, among others.

As FIG. 7 also indicates, the bottom row of stimuli includes an example where each presented stimulus corresponds to or matches the compound attribute: “identical color and distinct quantity”, specifically, a pair of open blue triangles, a triplet of nested blue triangles, and a single solid blue triangle, each of which corresponds to both of these attributes (of the compound attribute). As with the top row of example, stimuli, these stimuli may also match various other attributes, e.g., “identical shape and distinct texture”, and “distinct quantity”, among others.

In one embodiment, the stimuli may be restricted in form and/or content to some specified set of stimuli. For example, the at least one target attribute may be restricted to a subset of shape, color, texture, and quantity or number, as with exemplary Task 1 (where quantity is not a considered attribute), and each stimulus in the stimulus stream may be restricted to a specified set of shapes or figures, each with a specified subset of attributes. Note however, that in different embodiments, any sets of shapes and attributes (or numbers of streams and numbers of attributes) may be used as desired.

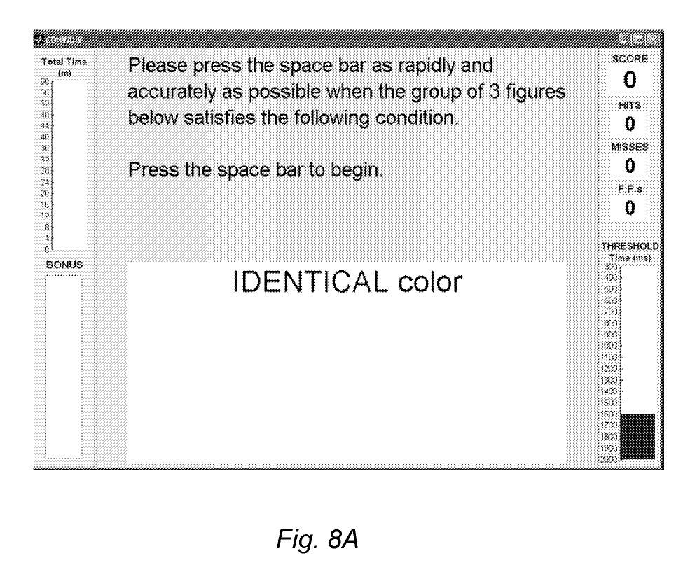

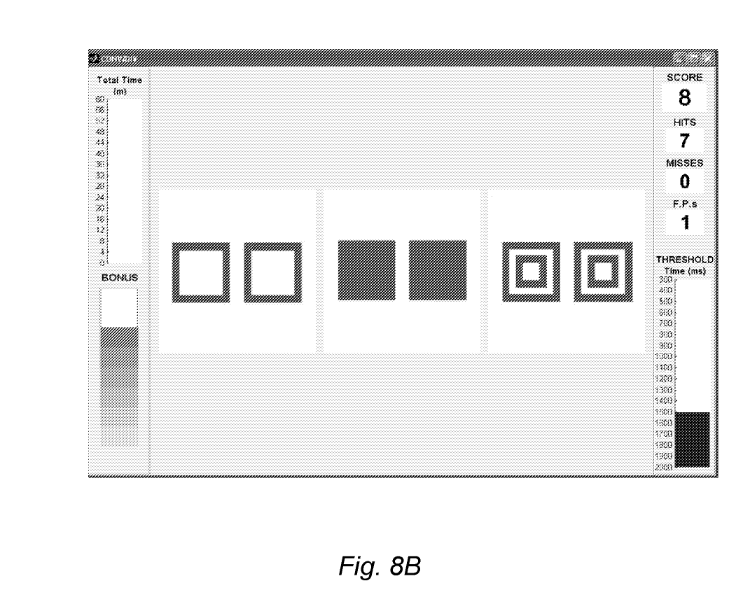

As noted above, in preferred embodiments, the exercises or tasks described herein are performed via a graphical user interface (GUI). As with Task 1 (or others), in one embodiment, instructions may be presented to the user prior to 303, e.g., explaining to the subject what to expect, and what is expected of the subject in the exercise or task. FIG. 8A is a screenshot of an exemplary GUI for the example Task 2, according to one embodiment. As FIG. 8A shows, in this embodiment, instructions are presented indicating that in this task or exercise, the subject is to press the space bar as quickly and accurately as possible when a group of stimuli (e.g., a group of figures, in this example, three stimulus groups, each with one or more shapes) is displayed that matches or corresponds to the at least one target attribute. As may be seen, the GUI also displays the at least one target attribute, in this case: “IDENTICAL color”, meaning that the subject is to respond positively (hitting the space bar) whenever all the stimuli in a stimulus group shown have the same color.

FIG. 8B is a screenshot of an exemplary GUI according to the example Task 1 described above, where the stimulus group presented includes three pair of red squares, each with a different (distinct) texture, which clearly corresponds to or matches the target attribute “IDENTICAL color”. Note that this stimulus group corresponds to three stimulus streams, each providing a pair of red squares (but of distinct textures-open, solid, and nested).

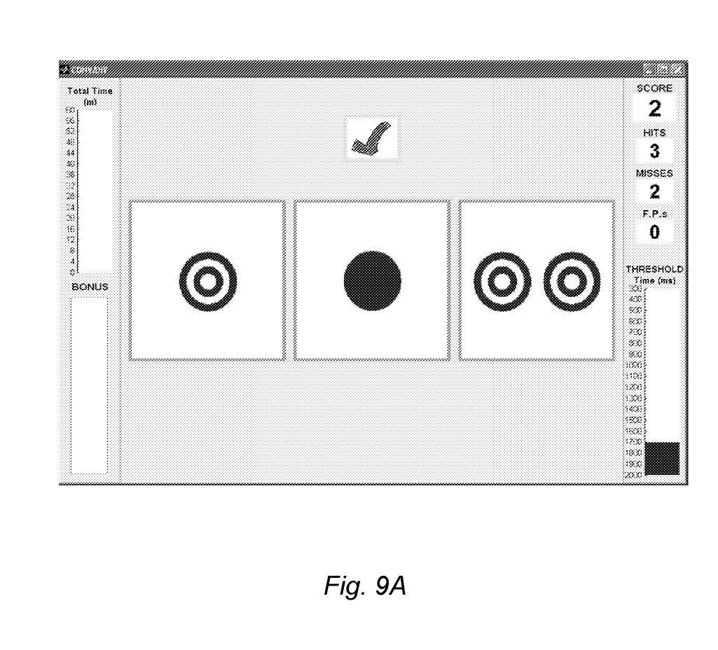

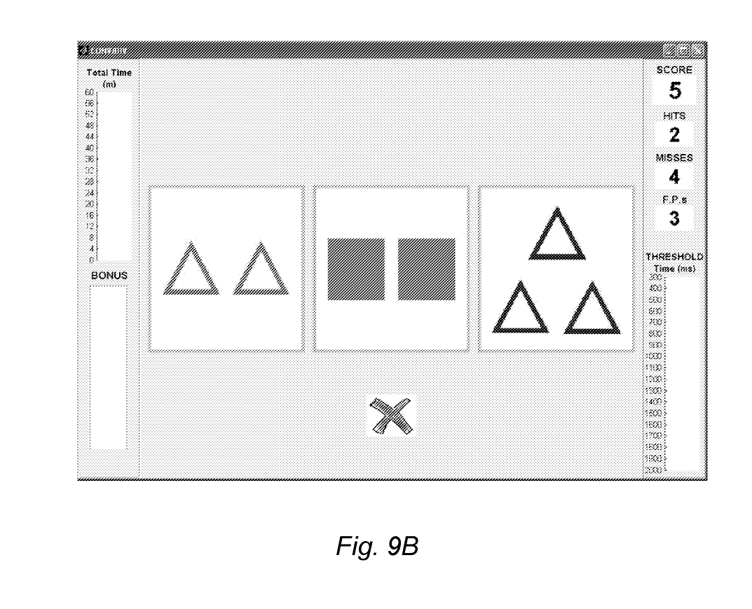

FIG. 9A illustrates another screenshot of an exemplary GUI for Task 2. In this example, the stimulus group includes a blue open circle, a blue solid circle, and a pair of nested blue circles. A possible matching or corresponding attribute for this stimulus group might be “IDENTICAL shape and IDENTICAL color”, among others. Apparently, the subject has correctly identified this match, as a “check” is displayed, indicating a correct response.

FIG. 9B illustrates a further screenshot of an exemplary GUI for Task 2. In this example, the stimulus group includes a pair of open green triangles, a pair of solid green squares, and a triplet of open blue triangles. Apparently, the subject has incorrectly identified this as a match, as an “X” is displayed, indicating an incorrect response. Note that this particular stimulus group does not match any of the example relationship attributes described above, e.g., identical color, number, shape, or texture, nor distinct color, number, shape, or texture.

Summarizing some aspects of the exemplary task described above, each stimulus group includes a plurality of stimuli, and the at least one target attribute may include one or more relationships of attributes of the plurality of stimuli, each relationship including one or more of: identical, wherein each stimulus of the plurality of stimuli has a common attribute value, or distinct, where each stimulus of the plurality of stimuli has a different attribute value; and where each of the plurality of stimuli includes a plurality of attributes, including two or more of: color, shape, texture, or quantity, absence of a specified color, absence of a specified shape, absence of a specified texture, or absence of a specified quantity. Of course, other attributes and/or target attributes may be used as desired.

As noted above, in some embodiments, the exercise or task may include various levels through which the subject may progress as the exercise or task is performed. One example of level criteria for Task 2 may include:

Level 1: Identical—single identical attribute;

Level 2: Distinct—single distinct attribute;

Level 3: Combination of two identical or distinct attributes;

Level 4: Combination of three identical or distinct attributes; and

Level 5: Combination of four identical or distinct attributes.

It should be noted, however, that any other level criteria may be used as desired, the above being exemplary only.

Thus, in various embodiments, the method may include one or more tasks or exercises where at least one target attribute is presented, including one or more relationships among attributes of stimuli, after which multiple streams of stimuli (i.e., where each stimulus group includes multiple elements) are presented, and the user indicates when the stimuli match or correspond to the specified target attribute(s). As noted above, however, other or additional attributes (including relationships) may be used as desired. For example, in some embodiments, compound attributes may be conjoined with other logical operands than simply “AND”, such as, for example, “NOT”, “OR”, or even “XOR” (exclusive OR), among others, e.g., “IDENTICAL color or DISTINCT texture”, “IDENTICAL shape and not IDENTICAL color”, and so forth.

In some embodiments, the exercise may include a combination of the above tasks (possibly including other continuous performance tasks, as well). For example, in one embodiment, the presenting at least one target attribute (303), presenting the continuous sequence of stimulus groups (304), and the requiring, determining, and adjusting (306) may compose a session. The repeating 314 (i.e., the repeating of 314 a plurality of times) may include two or more of: performing one or more sessions where the one or more stimuli include a plurality of attributes, including two or more of: color, shape, or texture, and where the at least one target attribute includes one or more of: color, shape, texture, absence of a specified color, absence of a specified shape, or absence of a specified texture, among others; and performing one or more sessions where each stimulus group includes a plurality of stimuli, where the at least one target attribute includes one or more relationships of attributes of the plurality of stimuli, each relationship including one or more of: identical, wherein each stimulus of the plurality of stimuli has a common attribute value, or distinct, wherein each stimulus of the plurality of stimuli has a different attribute value, and each of the plurality of stimuli including a plurality of attributes, including one or more of: color, shape, texture, or quantity, absence of a specified color, absence of a specified shape, absence of a specified texture, or absence of a specified quantity, among others.

In some embodiments, the repeating of 314, and/or the performing the repeating of 314 a plurality of times, may occur a specified number of times each day, for a specified number of days. In other words, the subject may perform a plurality of sessions each day over a period of days, e.g., for 6 months, to improve cognition.

In some embodiments, certain information may be maintained and recorded over the course of the exercise. For example, in one exemplary embodiment, the following information may be recorded: the name of the subject; the age of the subject; the gender of the subject; the number of trial groups completed; all scores achieved during the exercise; the conditions in force for each trial group; time/date for each session; and time spent on each trial group, among others. Of course, this information is meant to be exemplary only, and other information may be recorded as desired.

In some embodiments, the method may also include performing one or more practice sessions, i.e., prior to performing the method elements described above. For example, in some embodiments, one or more practice sessions may be performed prior to the beginning of training to familiarize the subject with the nature and mechanisms of the exercise. In some embodiments, in each practice session, a specified number of trials (e.g., 1) for each of one or more practice conditions may be performed. In some embodiments, the subject may be able to invoke such practice sessions at will during the exercise, e.g., to re-familiarize the subject with the task at hand.

Graphical User Interface

As discussed above, in preferred embodiments, the exercises described herein are performed via a graphical user interface (GUI), examples of which are shown in FIGS. 6A-6C, 8A-8B, and 9A-9B, although it should be noted that the GUI appearance and functionalities described herein are meant to be exemplary only, and are not intended to limit the GUIs to any particular form, function, or appearance.

As described above, in one embodiment, the GUI may include an introductory screen presenting task-specific instructions to the subject, as shown in FIGS. 6A and 8A. The user may invoke initiation of the exercise when ready, e.g., by pressing the space bar. In preferred embodiments, the GUI may include various indicators that may operate to indicate the subjects progress and/or performance in the exercise or task. For example, turning again to FIG. 6B, as may be seen, in this embodiment, the GUI includes a stimulus display area (here shown displaying a red open circle). The indicators shown include a total time indicator, labeled “Total Time”, indicating the time the subject has spent so far in the exercise, a bonus meter or indicator, labeled “BONUS”, that may indicate how close the subject is to achieving a bonus award (e.g., bonus points), a score indicator, labeled “SCORE”, indicating the current points achieved in the exercise, hit and miss indicators, respectively indicating the number of hits (i.e., true positives) and misses (i.e., false negatives), a false positive indicator, labeled “F.P.s”, indicating the number of false positives, and a threshold meter or indicator, labeled “THRESHOLD”, indicating the current value of the duration and/or ISI in the exercise. Of course, in other embodiments, other indicators may be used as desired.

In one embodiment, following a specified delay, e.g., 2000 ms, two or more streams of stimuli may be presented continuously, as described above. The duration and ISI of the presentation may be randomly chosen, determined based on prior studies, or based on past performances of the exercise, among other initialization techniques.

The following relates the subject's responses, described above, to aspects of the GUI, particularly the trial-by-trial rewards presented by or via the GUI, according to one embodiment, although it should be noted that these awards are meant to be exemplary only. For example, note that the responses described below are with respect to embodiments where the subject is to inhibit responses to non-targets. Embodiments where the subject is to respond respectively to targets and non-targets may use similar rewards.

Trial-by-Trial Rewards

Hit (true positive): When the subject's response is a hit or true positive, meaning that the subject has correctly indicated that the stimuli in a stimulus group correspond to or match the at least one target attribute, the subject may be rewarded with auditory feedback, e.g., a success sound (e.g., a “ding”), visual feedback (e.g., a graphical success indication), addition of points, and/or bonus meter advances.

Non-response (true negative): When the subject's response is a non-response or true negative, meaning that the subject has correctly refrained from indicating correspondence or matching for the stimulus group, the subject may be rewarded with bonus meter advances, and after five non-responses in a row, may be rewarded with auditory feedback, e.g., a success sound (e.g., a “ding”), visual feedback (e.g., a graphical success indication), and addition of points.

As described above, FIGS. 6B and 9A illustrate exemplary screenshots of the GUI presenting visual feedback for a correct response, i.e., a hit, or a non-response, in this case, a checkmark presented above the visual stimulus, although it should be noted that any other visual feedback may be used, in addition to other rewards.

False positive: When the subject's response is a false positive, meaning that the subject has incorrectly indicated that the stimuli in a stimulus group correspond to or match the at least one target attribute, the subject may be rewarded (penalized) with auditory feedback, e.g., an error sound (e.g., a “thunk”), visual feedback (e.g., a graphical indication of error or failure), bonus meter reset (where progress toward a bonus is reset to zero or decreased).

Miss (false negative): When the subject's response is a false negative, meaning that the subject has incorrectly failed to indicate that the stimuli in a stimulus group correspond to or match the at least one target attribute, the subject may be rewarded (penalized) with a bonus meter reset (where progress toward a bonus is reset to zero or decreased), and/or a frame color change, i.e., the GUI may modify the color of the region around the visual stimulus to indicate an error. Other rewards or penalties may be used as desired, e.g., visual feedback, etc., e.g., an “X” under the stimulus, resetting the bonus meter, and so forth.

FIGS. 6C and 9B illustrate exemplary screenshots of the GUI presenting visual feedback for an incorrect response. As shown, in this case, the GUI has modified the frame color (around the visual stimulus), presented visual feedback in the form of an “X” under the visual stimulus, and reset the bonus meter.

Threshold Determination

As indicated above, in some embodiments, the duration and/or ISI may be adjusted using an adaptive procedure. For example, in some embodiments, the duration and/or ISI may be adjusted using a maximum likelihood procedure. Such procedures may be used to modify or set an adjustable attribute (or combination of attributes) of a presented stimulus, whereby trials in the task or exercise may be made more or less difficult. Such an adjustable parameter is generally referred to as a stimulus intensity, and the maximum likelihood procedure is used to determine a stimulus threshold, which is the value of the stimulus intensity at which the subject achieves a specified level of success, e.g., 0.9, corresponding to a 90% success rate. There are various approaches whereby such thresholds may be assessed or determined, such as, for example, the well known QUEST (Quick Estimation by Sequential Testing) threshold method, which is an adaptive psychometric procedure for use in psychophysical experiments, or a related method, referred to as the ZEST (Zippy Estimation by Sequential Testing) procedure or method, among others.

Exercise based threshold determination may be designed to assess a subject's threshold with respect to stimuli on a given exercise, and can be used to adjust stimulus presentation to achieve and maintain a desired success rate for the subject, e.g., with respect to a particular exercise, task, and/or condition. In preferred embodiments of the exercises and tasks described herein, the stimulus intensity is the duration and/or ISI of the presented stimuli. In other words, the progressions (successive modifications or adjustments of presentation parameters, e.g., duration and/or ISI) in the exercise may be calculated using adaptive procedure, e.g., a maximum likelihood procedure, e.g., the ZEST procedure. For example, for each trial, a likelihood function may be calculated (based on the subject's response) to determine the next best guess of the true threshold. This estimate may be used (and possibly displayed) in the next trial. Based on the trial outcome and all previous trials the next best guess may be calculated and used in the presentation of stimuli for the next trial (and possibly displayed). As the probability function narrows and the standard deviation decreases, the estimate of true threshold approaches the true value—e.g., achieving a “good enough” threshold value. Note that the ZEST procedure is a modification of the QUEST procedure—the ZEST procedure uses the mean while the QUEST procedure uses the mode. In preferred embodiments, when training, a single stair ZEST procedure may be used, e.g., with a threshold level of 85%, although other values may be used as desired.

In preferred embodiments, the maximum likelihood procedure may be a continuous performance maximum likelihood procedure (as opposed to a discrete performance maximum likelihood procedure), such as a continuous performance ZEST procedure. One such a procedure, referred to as a continuous performance task (CPT), is described below.

Continuous Performance Progression

The Continuous Performance Task (CPT) is a neuropsychological task or exercise that consists of a series of stimulus groups presented one after another. The subject may be instructed to attend to a particular stimulus or a category of stimuli and respond to the presented stimulus group. The initial duration of CPT, e.g., the initial value of the presentation time for the stimulus groups, may differ from task to task.

Subject Response

In preferred embodiments, there are four possible response outcomes: true positive, true negative, false positive and false negative, defined as follows:

Correct responses:
True positive: hit or correct response to a correct target, or
True negative: no response to a non-target (or correct response to a non-target).

Incorrect responses:
False negative: miss or no response to a correct target, or
False positive: response to a non-target (or incorrect response to a non-target).
   Training at Optimal Level

Continuous Performance Tasks have traditionally been conducted where the stimulus Onset Asynchrony (SOA) time, e.g., the duration plus the ISI, is fixed throughout a session or is only altered after a block of stimuli is presented. Because the appropriate SOA time may differ greatly depending on: 1) the nature of the task; 2) the type of stimulus set used; and 3) the ability of the participant, a progression algorithm that optimizes the training experience is desired, especially when the training of speed of processing is crucial. For example, if the SOA time is too long, the task will not challenge the participants and thus not engage them. If the SOA time is too short, participants may find the task frustrating and the task may lack training value. Thus, a new SOA time progression scheme is presented herein that optimizes training experience by changing the SOA from trial to trial using an adaptive procedure, e.g., based on a maximum likelihood method. This scheme is used in preferred embodiments of the continuous performance cognitive training exercises described above.

In one embodiment, the progression scheme employed is based on the ZEST (Zippy Estimation by Sequential Testing) Bayesian adaptive estimator method, which is a probabilistic procedure where a prior estimate value of a variable is updated sequentially via a likelihood function that contains all the previous trials' information, yielding a posterior estimate value that encompasses all the data generated so far from the initial assumptions to all the user's responses. In other embodiments, other adaptive procedures may be used, e.g., QUEST.

Psychometric Function

The procedure may begin with a pre-determined psychometric function, which describes the relationship between a parameter of a stimulus and the behavior of a person's response about a certain attribute of that stimulus. The psychometric function is generally a sigmoidal function, with the percentage of correct responses plotted against the stimulus parameter.

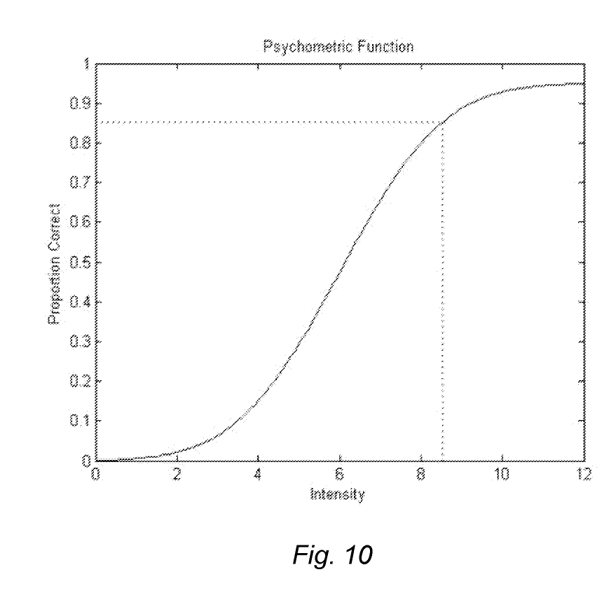

FIG. 10 illustrates an exemplary psychometric function based on a cumulative Gaussian distribution for a detection (yes/no) task with a 5% lapsing rate, and a target performance rate of 85%. In this example, the difficulty of the task decreases as the intensity parameter increases, as shown by the higher percent correct for higher parameter values. The dotted lines, at ˜8.5 intensity and ˜0.85 proportion correct, indicate the correspondence between the intensity parameter and the 85% performance rate.

The method makes several assumptions about the psychophysics:

1. The psychometric function has the same shape, except a shift along the stimulus intensity axis to indicate different performance values.

2. The performance value does not change from trial to trial.

3. Individual trials are statistically independent.

It should be noted, however, that in preferred embodiments, the methods described herein may be robust to violations of these rules, particularly to rules 2 and 3.

Prior Probability Density Function

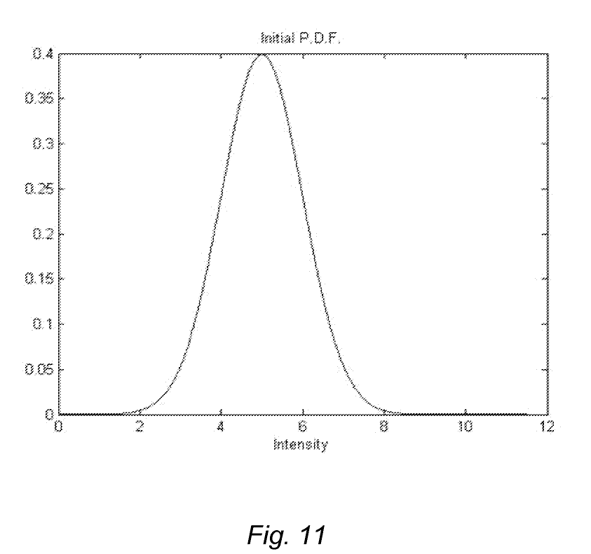

The prior probability density function (P.D.F.) is the initial distribution of the intensity values that yield the performance level in the psychophysical task. Since the function is a P.D.F., a probability function, it is a nonnegative function with the area under the curve summing up to 1, i.e., the total probability equals 100%. Typical examples of prior P.D.F.s are Gaussian distributions, Poisson distributions, Weibull distributions, and rectangular distributions, although other distributions may be used as desired. The initial estimate of intensity is often taken to be the mean of the P.D.F. FIG. 11 illustrates an exemplary probability density function with initial intensity estimate x=5, indicated by the peak of the function.

Likelihood Function

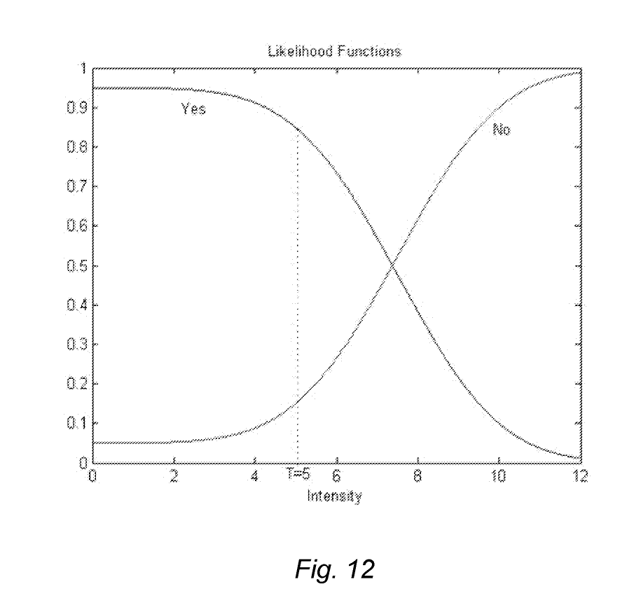

The prior P.D.F. may be adjusted after each trial by one of two likelihood functions, which are the respective probability functions describing the subject's likelihood of responding “yes” or “no” to the stimulus at the intensity as a function of the intensity. Since the psychometric function has a constant shape and the form F(x−T), fixing the intensity x and treating intensity T as the independent variable, the “yes” likelihood, p=F(−(T−x)), is thus the mirror image of the psychometric function about T, and the “no” likelihood function is then simply 1−p. FIG. 12 illustrates exemplary likelihood functions with T=5 and an 85% correct rate, correspondingly labeled “Yes” and “No”.

Posterior Probability Density Function

After a presentation is complete and the response is noted, the prior P.D.F. may be updated using Bayes' rule, by multiplying the prior P.D.F. by the likelihood function corresponding to the subject's response to the trial's stimulus intensity to obtain the posterior P.D.F. The mean of the posterior P.D.F. may then be used as the new intensity estimate. This procedure may be repeated after every presentation to determine the subsequent intensity of the stimulus.

Terminating Condition

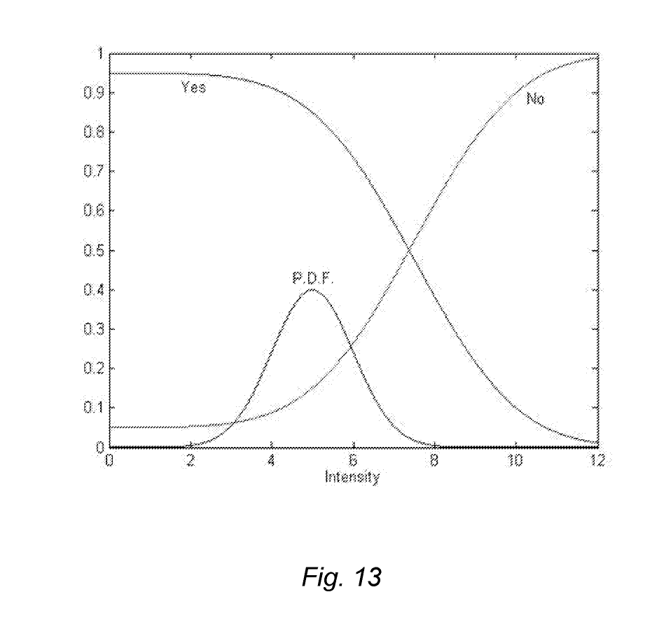

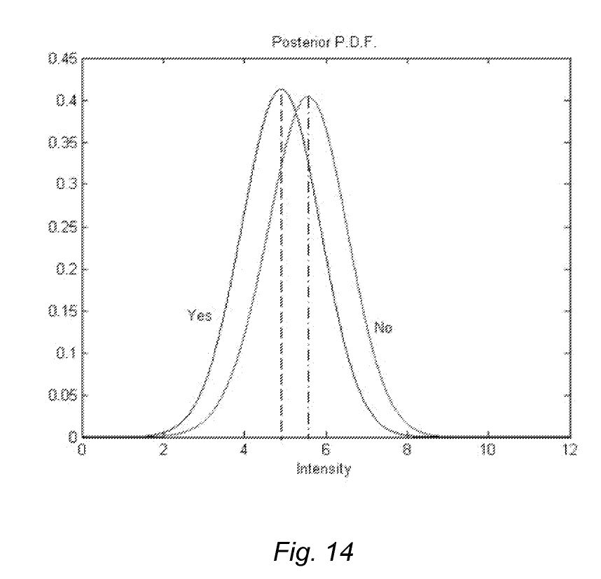

The procedure may be terminated if the posterior P.D.F. satisfies a certain confidence level. This may first require the posterior P.D.F. computed to be normalized. If the confidence interval of the posterior P.D.F. yielding the confidence level is less than the specified length, then the procedure may be terminated, and the final intensity obtained is the threshold intensity. FIG. 13 illustrates an exemplary prior P.D.F. (labeled “P.D.F.”) superimposed with exemplary likelihood functions (again, labeled “Yes” and “No”). FIG. 14 illustrates an exemplary posterior P.D.F. with Yes/No response. Note that the intensity for the respective Yes and No P.D.F. functions differs.

CPT Framework

In one embodiment of the continuous performance framework described herein, illustrated by FIGS. 10-14, an SOA time (represented by intensity) is sought that results in the subject achieving an 85% correct rate, with the initial SOA time set at 5 units. In this example, if the response to the trial is correct, the next SOA time is updated to ˜4.8, while an incorrect response yields subsequent SOA time ˜5.5. This new SOA time may then be used for the presentation duration of the next stimulus (e.g., stimulus group) in the block or sequence (stream). The procedure may continue in the same fashion for the updated SOA time to yield the subsequent SOA time for the next presentation. Note, however, that these particular values are meant to be exemplary only, and that any other values may be used as desired.

Thus, various embodiments of the methods disclosed herein may utilize an adaptive procedure, e.g., a continuous performance maximum likelihood procedure, e.g., continuous performance ZEST, to adaptively modify one or more stimulus presentation parameters, e.g., duration and/or ISI, in continuous performance tasks or exercises utilizing stimulus streams.

It should also be noted that the particular exercises and tasks disclosed herein are meant to be exemplary, and that other continuous performance cognitive training exercises using stimulus streams may be used as desired, possibly in combination. In other words, the exercises and tasks described herein are but specific examples of cognitive training exercises and tasks using a computing system to present stimulus streams to a subject, record the subject's responses, and modify some aspect of the stimuli based on these responses, where these method elements are repeated in an iterative manner using multiple sets of stimuli to improve the subject's cognition. Note particularly that such cognitive training using a variety of such stimulus stream-based exercises or tasks, possibly in a coordinated manner, is contemplated. Thus, various embodiments of the cognitive training exercises and tasks described herein may be used singly or in combination to improve the subject's cognitive skills.

Those skilled in the art should appreciate that they can readily use the disclosed conception and specific embodiments as a basis for designing or modifying other structures for carrying out the same purposes of the present invention without departing from the spirit and scope of the invention as defined by the appended claims. For example, various embodiments of the methods disclosed herein may be implemented by program instructions stored on a memory medium, or a plurality of memory media.

We claim:

1. A computer-implemented method for enhancing cognition in a subject, utilizing a computing device to present stimuli and to receive responses from the subject, the method comprising:
a) providing a set of stimuli for presentation to the subject;
b) presenting at least one target attribute to the subject;
c) presenting a continuous sequence of stimulus groups from the set of stimuli to the subject one stimulus group at a time, wherein each stimulus group comprises one or more stimuli, wherein each stimulus group is presented for a specified duration, and wherein the stimulus groups in the continuous sequence of stimulus groups are separated by a specified inter-stimulus-interval (ISI);
d) for each stimulus group in the continuous sequence of stimulus groups,
requiring the subject to respond to the stimulus group by indicating when the one or more stimuli in the stimulus group correspond to the at least one target attribute;
determining if the subject responded correctly for the stimulus group;
adjusting the duration and/or the ISI based on said determining using an adaptive procedure; and
e) repeating b)-d) one or more times in an iterative manner to improve the cognition of the subject.

2. The method of claim 1, wherein b)-d) is performed under a specified condition, wherein the condition specifies one or more attributes of said presenting the continuous sequence of stimulus groups, the method further comprising:
repeating b)-e) a plurality of times in an iterative manner, wherein, for each iteration, b)-e) is performed under a respective condition.

3. The method of claim 2, wherein each condition specifies one or more of:
length of the continuous sequence of stimulus groups;
length of time of said presenting the continuous sequence of stimulus groups;
correspondence frequency, comprising a ratio of stimulus groups in which the one or more stimuli in the stimulus group correspond to the at least one target attribute to stimulus groups in which the one or more stimuli in the stimulus group do not correspond to the at least one target attribute; or
target/foil confusability, comprising a degree to which stimuli that correspond to the at least one target attribute are similar to stimuli that do not correspond to the at least one target attribute.

4. The method of claim 1, further comprising:
indicating whether the subject responded correctly, wherein said indicating is performed audibly and/or visually.

5. The method of claim 4, further comprising:
requiring the subject to respond to the stimulus group by refraining from indicating when the one or more stimuli in the stimulus group do not correspond to the at least one target attribute.

6. The method of claim 5, wherein the subject's response to the stimulus group comprises one of:
a correct response, comprising:
a true positive, wherein the subject correctly indicates when the one or more stimuli in the stimulus group correspond to the at least one target attribute; or
a true negative, wherein the subject correctly refrains from indicating when the one or more stimuli in the stimulus group do not correspond to the at least one target attribute; or
an incorrect response, comprising:
a false negative, wherein the subject fails to indicate when the one or more stimuli in the stimulus group correspond to the at least one target attribute; or
a false positive, wherein the subject incorrectly indicates that the one or more stimuli in the stimulus group correspond to the at least one target attribute.

7. The method of claim 4, further comprising:
requiring the subject to respond to the stimulus group by indicating when the one or more stimuli in the stimulus group does not correspond to the at least one target attribute.

8. The method of claim 7, wherein the subject's response to the stimulus group comprises one of:
a correct response, comprising:
a true positive, wherein the subject correctly indicates when the one or more stimuli in the stimulus group correspond to the at least one target attribute; or
a true negative, wherein the subject correctly indicates when the one or more stimuli in the stimulus group do not correspond to the at least one target attribute; or
an incorrect response, comprising:
a false negative, wherein the subject incorrectly indicates that the one or more stimuli in the stimulus group do not correspond to the at least one target attribute; or
a false positive, wherein the subject incorrectly indicates that the one or more stimuli in the stimulus group correspond to the at least one target attribute.

9. The method of claim 6, wherein said requiring the subject to respond comprises requiring the subject to respond before a next stimulus group is presented.

7. The method of claim 4, wherein said indicating whether the subject responded correctly comprises:
rewarding the subject if a specified level of success is achieved; or
penalizing the subject if a specified level of failure is achieved;
wherein the rewarding and penalizing each comprises one or more of:
auditory feedback;
visual feedback;
point modification; or
change in bonus status.

8. The method of claim 4, further comprising:
terminating said repeating b)-d) if the subject responds incorrectly a specified number of times consecutively.

9. The method of claim 1, wherein b)-d) composes a session, the method further comprising:
presenting a reward to the subject at the end of each session, wherein the reward is presented visually and/or audibly.

10. The method of claim 1, wherein the adaptive procedure comprises a continuous performance maximum likelihood procedure.

11. The method as recited in claim 10, wherein the continuous performance maximum likelihood procedure comprises one or more of:
a continuous performance QUEST (quick estimation by sequential testing) threshold procedure; or
a continuous performance ZEST (zippy estimation by sequential testing) threshold procedure.

12. The method of claim 1, wherein said adjusting the duration and/or the ISI comprises:
adjusting the duration and/or the ISI to approach and substantially maintain a specified success rate for the subject.

13. The method of claim 12, wherein said adjusting the duration and/or the ISI to approach and substantially maintain a specified success rate for the subject uses a single stair continuous performance maximum likelihood procedure.

14. The method of claim 1, wherein b)-d) compose a session, and wherein for each session, an initial value of the duration and/or the ISI and a final value of the duration and/or the ISI are averaged to determine the initial value of the duration and/or the ISI for the next session.

15. The method of claim 1, wherein b)-d) compose a session, the method further comprising:
for each session, averaging reaction times for each stimulus group in the continuous sequence of stimulus groups to determine a minimum value for the duration and/or the ISI for the next session, wherein each reaction time comprises a respective delay between the presentation of each stimulus group in which the one or more stimuli in the stimulus group correspond to the at least one target attribute and the subject's response to the stimulus group.

16. The method of claim 1, wherein each stimulus group presentation and corresponding subject response composes a trial, the method further comprising:
for each trial, recording one or more of:
the at least one target attribute;
the stimulus group;
whether or not the stimuli in the stimulus group corresponds to the at least one target attribute;
the duration;
the ISI;
the subject's response;
the correctness or incorrectness of the subject's response;
a reaction time for the trial, comprising the delay between the presentation of each stimulus group in which the stimuli correspond to the at least one target attribute and the subject's response to the stimulus group; or
statistical measures for the adaptive procedure.

17. The method of claim 1, wherein the continuous sequence of stimulus groups comprises one or more continuous stimulus streams, wherein the one or more stimuli in each stimulus group are from respective continuous stimulus streams of the one or more continuous stimulus streams.

18. The method of claim 1, wherein the one or more stimuli comprise a plurality of attributes, comprising two or more of:
color;
shape; or
texture.

19. The method of claim 1, wherein the at least one target attribute comprises one or more of:
color;
shape;
texture;
absence of a specified color;
absence of a specified shape; or
absence of a specified texture.

20. The method of claim 1, wherein each stimulus group comprises a plurality of stimuli.

21. The method of claim 20, wherein the continuous sequence of stimulus groups comprises a plurality of continuous stimulus streams, wherein the plurality of stimuli in each stimulus group are from respective continuous stimulus streams of the plurality of continuous stimulus streams.

22. The method of claim 20, wherein each of the plurality of stimuli comprises a plurality of attributes, comprising two or more of:
color;
shape;
texture; or
quantity.

23. The method of claim 20, wherein the at least one target attribute comprises one or more relationships of attributes of the plurality of stimuli, each relationship comprising one or more of:
identical, wherein each stimulus of the plurality of stimuli has a common attribute value; or
distinct, wherein each stimulus of the plurality of stimuli has a different attribute value;
wherein the attributes of the plurality of stimuli comprises two or more of:
color;
shape;
texture;
quantity;
absence of a specified color;
absence of a specified shape;
absence of a specified texture; or
absence of a specified quantity.

24. The method of claim 1, wherein b)-d) composes a session, the method further comprising:
repeating b)-e) a plurality of times in an iterative manner, wherein said repeating comprises two or more of:
performing one or more sessions wherein:
each of the one or more stimuli comprises a plurality of attributes, comprising two or more of:
color;
shape; or
texture; and
the at least one target attribute comprises one or more of:
color;
shape;
texture;
absence of a specified color;
absence of a specified shape; or
absence of a specified texture; and
performing one or more sessions wherein:
each stimulus group comprises a plurality of stimuli;
wherein the attributes of the plurality of stimuli comprises two or more of:
color;
shape;
texture;
quantity;
absence of a specified color;
absence of a specified shape;
absence of a specified texture; or
absence of a specified quantity; and
the at least one target attribute comprises one or more relationships of attributes of the plurality of stimuli, each relationship comprising one or more of:
identical, wherein each stimulus of the plurality of stimuli has a common attribute value; or
distinct, wherein each stimulus of the plurality of stimuli has a different attribute value.

25. The method of claim 1, further comprising:
repeating b)-e) a plurality of times in an iterative manner, wherein said repeating b)-e) occurs a specified number of times each day, for a specified number of days.

26. The method of claim 1, wherein b)-d) compose a session, the method further comprising:
performing an initial session prior to performing b)-d), wherein the initial session comprises b)-d), but wherein, in performing d) in the initial session, neither the duration nor the ISI is adjusted.

27. A computer accessible memory medium comprising program instructions for enhancing cognition in a subject, utilizing a computing device to present stimuli and to receive responses from the subject, wherein the program instructions are executable by a processor to perform:
a) providing a set of stimuli for presentation to the subject;
b) presenting at least one target attribute to the subject;
c) presenting a continuous sequence of stimulus groups from the set of stimuli to the subject one stimulus group at a time, wherein each stimulus group comprises one or more stimuli, wherein each stimulus group is presented for a specified duration, and wherein the stimulus groups in the continuous sequence of stimulus groups are separated by a specified inter-stimulus-interval (ISI);
d) for each stimulus group in the continuous sequence of stimulus groups,
requiring the subject to respond to the stimulus group by indicating when the one or more stimuli in the stimulus group correspond to the at least one target attribute;
determining if the subject responded correctly for the stimulus group;
adjusting the duration and/or the ISI based on said determining using an adaptive procedure; and
e) repeating b)-d) one or more times in an iterative manner to improve the cognition of the subject.
